(19) United States
(12) Patent Application Publication
(10) Pub. No.: US 2023/0054898 A1
(43) Pub. Date: Feb. 23, 2023

(54) KINK-RESISTANT EXPANDABLE SHEATH

(71) Applicant: Edwards Lifesciences Corporation, Irvine, CA (US)

(72) Inventors: Nikolai Gurovich, Hadera (IL); Roy Shitrit, Zichron Yaakov (IL); Itay Avinathan, Tel-Aviv (IL)

(21) Appl. No.: 17/982,915

(22) Filed: Nov. 8, 2022

Related U.S. Application Data
(63) Continuation of application No. PCT/US2021/032271, filed on May 13, 2021.
(60) Provisional application No. 63/024,386, filed on May 13, 2020.

(51) Int. Cl. A61F 2/24; A61F 2/966

(52) U.S. Cl. CPC A61F 2/2436; A61F 2/966; A61M 2025/0024

(57) ABSTRACT

Expandable sheaths are disclosed herein. In some aspects, the sheath comprises at least one plurality of struts arranged to form an expandable cylindrical frame such that the formed frame is kink resistant. The disclosed frames are configured to expand from a first diameter d₁ in an unexpanded position to a second diameter d₂ an expanded position upon passage of a medical device, while maintaining the kink resistance. Also disclosed are methods of making the same.

### CROSS-REFERENCE TO RELATED APPLICATIONS

This application is a continuation of International Application No. PCT/US2021/032271, filed May 13, 2021, which claims the benefit of U.S. Provisional Application No. 63/024,386, filed May 13, 2020, each of the above-referenced applications are incorporated herein by reference in its entirety.

### FIELD

The present application relates to expandable introducer sheaths for prosthetic devices such as transcatheter heart valves and methods of making the same.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

An introducer sheath can be used to safely introduce a delivery apparatus into a patient's vasculature (e.g., the femoral artery). An introducer sheath generally has an elongated sleeve that is inserted into the vasculature and a housing that contains one or more sealing valves that allow a delivery apparatus to be placed in fluid communication with the vasculature with minimal blood loss. Such introducer sheaths may be radially expandable. However, such sheaths tend to have complex mechanisms, such as ratcheting mechanisms that maintain the sheath in an expanded configuration once a device with a larger diameter than the sheath's original diameter is introduced. Existing expandable sheaths can also be prone to the kinks as a consequence of the application of longitudinal force attendant to passing a prosthetic device through the sheath, especially during passing through a curved vasculature of various radii. As a result, an increased force is required to insert the prosthetic device through the narrowed sheath.

Accordingly, there remains a need in the art for an improved introducer sheath for endovascular systems used for implanting valves and other prosthetic devices. Still, further, there is a need for introducer sheaths that exhibit a reduced insertion force as compared to conventional introducers. Also, there is still a need for methods of making the same.

### SUMMARY

The aspects of the present disclosure relate to the kink-resistant expandable sheaths for deploying a medical device. The expandable sheaths for deploying a medical device comprises at least one plurality of struts arranged to form an expandable cylindrical frame having a proximal and distal end, an inner surface and an outer surface, and a central axis extending along a length of the frame; wherein the expandable cylindrical frame has a first length and an opposite second length, and wherein, in a first position, at least a portion of the first length and at least a portion of the second length are substantially the same; wherein the expandable cylindrical frame is movable from the first position to a second position by curving away from the central axis, wherein at the second position at least a portion of the first length shortens and at least a portion of the second length elongates; wherein the inner surface of the expandable cylindrical frame defines a lumen configured to receive a medical device; and wherein the expandable cylindrical frame is configured to expand from a first diameter d₁ in an unexpanded position to a second diameter d₂ an expanded position upon passage of a medical device.

In yet other aspects, the portion of the first length and the portion of the second length in the second position are located along a curved portion of the frame. In yet further aspects, the first length is provided on a first portion of an outer diameter of the expandable cylindrical frame, and the second length is provided on a second portion of the outer diameter of the expandable cylindrical frame, wherein the first portion is circumferentially opposite to the second portion of the outer diameter.

In yet still further aspects, the expandable cylindrical sheath of any one of the disclosed aspects is kink resistant.

In some aspects, the plurality of struts can be arranged to form a zig-zag configuration comprising a plurality of substantially straight portions circumferentially followed by a plurality of substantially bend portions and wherein the zig-zag configuration is helically wound along a central axis to form the frame. In some exemplary aspects, each substantially straight portion of the plurality of substantially straight portions being of equal lengths. While in other exemplary aspects, the plurality of substantially straight portions can comprise one or more portions having unequal lengths at the distal and/or proximal ends of the frame.

Still further described herein are aspects where the plurality of struts are arranged in a zig-zag configuration to form a plurality of parallel circumferential rows, wherein each row of the plurality of parallel circumferential rows comprises a plurality of substantially straight portions circumferentially followed by a plurality of substantially bend portions; and wherein the plurality of parallel circumferential rows are axially coupled to each other by a singular vertical strut member disposed along a length of the frame of the medical device, the frame does not expand along the singular vertical strut member.

Also described herein are aspects where the plurality of struts are arranged in a configuration wherein each strut of the plurality of struts is able to individually elongate in a circumferential direction along the length of the frame upon introduction of the medical device.

Still further disclosed are the aspects where the plurality of struts are arranged in a zig-zag configuration to form a plurality of parallel circumferential rows, wherein each row of the plurality of parallel circumferential rows comprises a plurality of substantially straight portions circumferentially followed by a plurality of substantially bend portions; wherein adjacent rows of the plurality of parallel circumferential rows are coupled together by a singular bridging member extending from a substantially bend portion in a first row to a corresponding substantially bend portion in an adjacent second row, and wherein the bridging member coupling any two adjacent rows is circumferentially offset from a bridging member coupling any following two adjacent rows.

Also disclosed are the aspects where the cylindrical frame is formed by a first plurality of struts arranged along a first edge of a singular vertical member and a second plurality of struts arranged along an opposite second edge of the singular vertical member, wherein each strut of the first plurality of struts is spaced from each other such that a first gap is formed between two adjacent struts in the first plurality of struts; wherein each strut of the second plurality of struts is spaced from each other such that a second gap is formed between two adjacent struts in the second plurality of struts; wherein each strut of the first plurality of struts is axially offset along the singular vertical member from each strut of the second plurality of struts; and wherein in the unexpanded position, each strut of the first plurality of struts is disposed within the second gap, and each strut of the second plurality of struts is disposed within the first gap.

Still further, disclosed herein are the aspects where the sheath further comprises one or more polymeric layers. In such exemplary aspects, the polymeric layer comprises one or more of polyethylene, polypropylene, polyether block amide (PEBAX®), silicon, or any combination thereof.

In still further aspects, at least a portion of the frame is embedded within the one or more polymeric layers. In certain exemplary aspects, the one or more polymeric layers comprise a plurality of longitudinally extending folds wherein the sheath is at the first diameter d₁. While in other exemplary aspects, the longitudinally extending folds create a plurality of circumferentially spaced ridges and a plurality of circumferentially spaced valleys. In still further aspects, as the medical device passes through the sheath, the ridges and valleys can level out to allow the sheath to radially expand.

Methods of making expandable sheaths are also disclosed herein. One aspect of a method comprises arranging at least one plurality of struts to form an expandable cylindrical frame having a proximal and distal end, an inner surface and an outer surface, and a central axis extending along a length of the frame, wherein the expandable cylindrical frame has a first length and an opposite second length, and wherein in a first position at least a portion of the first length and at least a portion of the second length are substantially the same, wherein the expandable cylindrical frame is movable from the first position to a second position by curving away from the central axis, wherein at the second position at least a portion of the first length shortens and at least a portion of the second length elongates; wherein the inner surface of the expandable cylindrical frame defines a lumen configured to receive a medical device; and wherein the expandable cylindrical frame is configured to expand from a first diameter d₁ in an unexpanded position to a second diameter d₂ an expanded position upon passage of a medical device.

Still further disclosed are the aspects describing the methods further comprising a step of forming a pre-shaped flare at the distal end of the frame such that the pre-shaped flare is configured to stay compressed at the rest position and to assume a flared configuration upon passing the medical device. In such exemplary aspects, the pre-shaped flare is kept compressed by attaching a filament one or more of the plurality of struts at the distal end of the frame and extending it circumferentially around the plurality of struts at the distal end. In certain aspects, when the distal end of the frame is in the expanded position, the filament is configured to stay attached to one or more of the plurality of struts at the distal end of the frame without extending circumferentially around the plurality of struts at the distal end thereby allowing the distal end of the frame to flare radially outward.

It is understood that the methods disclosed herein include aspects where the medical device is a cardiovascular device. In such aspects, the cardiovascular device can include at least one of prosthetic heart valve and a stent. In still further aspects, disclosed herein methods comprise applying an outer layer on the sheath.

Additional aspects of the disclosure will be set forth, in part, in the detailed description, figures, and claims which follow, and in part will be derived from the detailed description or can be learned by practice of the invention. It is to be understood that both the foregoing general description and the following detailed description are exemplary and explanatory only and are not restrictive of the invention as disclosed.

### BRIEF DESCRIPTION OF THE DRAWINGS

FIG. 1 illustrates a delivery system for a cardiovascular prosthetic device, according to one exemplary aspect.

FIG. 2 illustrates an expandable sheath that can be used in combination with the delivery system of FIG. 1, according to one exemplary aspect.

FIG. 3 illustrates an expandable sheath in one exemplary aspect.

FIG. 4 is an exemplary frame in one exemplary aspect.

FIG. 5 is a magnified view of a distal portion of the frame of FIG. 4, according to one exemplary aspect.

FIG. 6 is a magnified view of a mid-portion of the frame of FIG. 4, according to one exemplary aspect.

FIG. 7 is a flattened view of a distal portion of the frame of FIG. 4, according to one exemplary aspect.

FIG. 8 is an exemplary frame of a distal portion of the frame in one exemplary aspect.

FIG. 9 is a magnified view of a mid-portion of the frame of FIG. 8, according to one exemplary aspect.

FIG. 10 is a flattened view of a distal portion of the frame of FIG. 8, according to one exemplary aspect.

FIG. 11 is a flattened view of a mid-portion portion of the frame of FIG. 9, according to one exemplary aspect.

FIG. 12 is an exemplary frame in one exemplary aspect.

FIG. 13 is a magnified view of a distal portion of the frame of FIG. 12.

FIG. 14 is a magnified view of a mid-portion of the frame of FIG. 12.

FIG. 15 is a flattened view of a distal portion of the frame of FIG. 13, according to one exemplary aspect.

FIG. 16 is an exemplary frame in one exemplary aspect.

FIG. 17 is a magnified view of a distal portion of the frame of FIG. 16.

FIG. 18 is a magnified view of a mid-portion of the frame of FIG. 16.

FIG. 19 is a flattened view of a distal portion of the frame of FIG. 17, according to one exemplary aspect.

FIG. 20A is an exemplary frame in one exemplary aspect.

FIG. 20B is a back perspective view of the frame of FIG. 20.

FIG. 21 is a flattened view of the frame of FIG. 20.

FIGS. 22A-22F depict simulated insertion and removal of a medical device through an exemplary frame of FIG. 20A in one exemplary aspect.

FIGS. 23A-23D depict simulated insertion and removal of a medical device through an exemplary frame of FIG. 20A in one exemplary aspect.

FIGS. 24A-24G depict simulated insertion and removal of a medical device through an exemplary frame of FIG. 20A in one exemplary aspect.

FIGS. 25A-25F depict simulated insertion and removal of a medical device through an exemplary frame of FIG. 20A in one exemplary aspect.

FIGS. 26A-26F depict simulated insertion and removal of a medical device through an exemplary frame of FIG. 20A in one exemplary aspect.

FIG. 27 are photographs of an exemplary sheath demonstrating kink resistance upon passing and removal of a medical device in conduits having a curve of the various radii.

FIG. 28 depicts an exemplary distal portion of a frame in one exemplary aspect with a filament circumferentially attached to the frame.

FIG. 29 depicts an exemplary distal portion of a frame in one exemplary aspect after the passage of a medical device.

FIGS. 30A-30B show exemplary ridges and valleys formed in an exemplary polymeric material.

### DETAILED DESCRIPTION

The present disclosure can be understood more readily by reference to the following detailed description, examples, drawings, and claims, and their previous and following description. However, before the present articles, systems, and/or methods are disclosed and described, it is to be understood that this disclosure is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the disclosure is provided as an enabling teaching of the disclosure in its best, currently known aspect. To this end, those skilled in the relevant art will recognize and appreciate that many changes can be made to the various aspects of the disclosure described herein while still obtaining the beneficial results of the present disclosure. It will also be apparent that some of the desired benefits of the present disclosure can be obtained by selecting some of the features of the present disclosure without utilizing other features. Accordingly, those of ordinary skill in the pertinent art will recognize that many modifications and adaptations to the present disclosure are possible and may even be desirable in certain circumstances and are a part of the present disclosure. Thus, the following description is again provided as illustrative of the principles of the present disclosure and not in limitation thereof.

### Definitions

As used in this application and in the claims, the singular forms “a,” “an,” and “the” include the plural forms unless the context clearly dictates otherwise. Thus, for example, reference to a “frame” includes aspects having two or more such frames unless the context clearly indicates otherwise.

It is also to be understood that the terminology used herein is for the purpose of describing particular aspects only and is not intended to be limiting. As used in the specification and in the claims, the term “comprising” can include the aspects “consisting of” and “consisting essentially of.”

Unless defined otherwise, all technical and scientific terms used herein have the same meaning as commonly understood by one of ordinary skill in the art to which this disclosure belongs. In this specification and in the claims, which follow, reference will be made to a number of terms that shall be defined herein.

For the terms “for example” and “such as,” and grammatical equivalences thereof, the phrase “and without limitation” is understood to follow unless explicitly stated otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”). As used herein, the term “and/or” includes any and all combinations of one or more of the associated listed items.

It will be understood that, although the terms “first,” “second,” etc., may be used herein to describe various elements, components, regions, layers and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of exemplary aspects.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein are interpreted accordingly.

As used herein, the term “substantially” means that the subsequently described event or circumstance completely occurs or that the subsequently described event or circumstance generally, typically, or approximately occurs.

Still further, the term “substantially” can in some aspects refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

In other aspects, as used herein, the term “substantially free,” when used in the context of a surface substantially free of defects, for example, is intended to refer to a surface that has less than about 5% of defects, less than about 4.5% of defects, less than about 4% of defects, less than about 3.5% of defects, less than about 3% of defects, less than about 2.5% of defects, less than about 2% of defects, less than about 1.5% of defects, less than about 1% of defects, less than about 0.5% of defects, less than about 0.1% of defects, less than about 0.05% of defects, or less than about 0.01% of defects of the total surface.

As used herein, the term “substantially,” in, for example, the context “substantially no change” refers to a phenomenon or an event that exhibits less than about 1% change, e.g., less than about 0.5%, less than about 0.1%, less than about 0.05%, or less than about 0.01% change.

As used herein, the term “substantially,” in, for example, the context “substantially identical” or “substantially similar” refers to a method or a system, or a component that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

As used herein, the term “atraumatic” is commonly known in the art and refers to a device or a procedure that minimized tissue injury.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

Although the operations of exemplary aspects of the disclosed method may be described in a particular, sequential order for convenient presentation, it should be understood that disclosed aspects can encompass an order of operations other than the particular, sequential order disclosed. For example, operations described sequentially may, in some cases, be rearranged or performed concurrently. Further, descriptions and disclosures provided in association with one particular aspect are not limited to that aspect and may be applied to any aspect disclosed.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

### Sheath

The expandable introducer sheaths described herein can be used to deliver a prosthetic device through a patient's vasculature to a procedure site within the body. The sheath can be constructed to be highly expandable and collapsible in the radial direction while limiting axial elongation of the sheath and, thereby, undesirable narrowing of the lumen. In still further aspects and as described herein, the sheath is substantially kink-free.

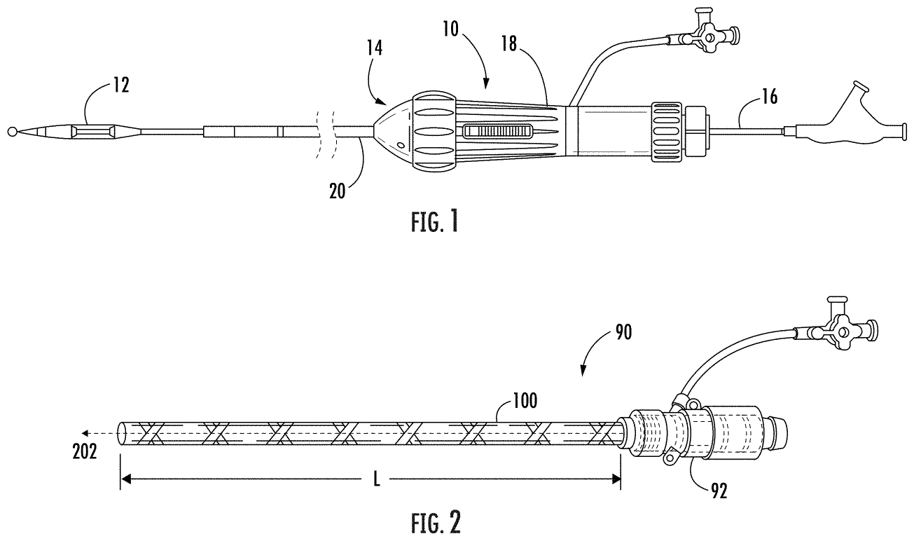

FIG. 1 illustrates a representative delivery apparatus 10 for delivering a medical device, such as a prosthetic heart valve or other prosthetic implant, to a patient. The delivery apparatus 10 is exemplary only and can be used in combination with any of the expandable sheaths as described herein. Likewise, the sheaths disclosed herein can be used in combination with any of the various known delivery apparatuses. The delivery apparatus 10 illustrated herein can generally include a steerable guide catheter 14 and a balloon catheter 16 extending through the guide catheter 14. A prosthetic device, such as a prosthetic heart valve 12, can be positioned on the distal end of the balloon catheter 16. The guide catheter 14 and the balloon catheter 16 can be adapted to slide longitudinally relative to each other to facilitate delivery and positioning of a prosthetic heart valve 12 at an implantation site in a patient's body. The guide catheter 14 includes a handle portion 18 and an elongated guide tube or shaft 20 extending from the handle portion 18.

The prosthetic heart valve 12 can be delivered into a patient's body in a radially compressed configuration and radially expanded to a radially expanded configuration at the desired deployment site. In some exemplary aspects, the prosthetic heart valve 12 is a plastically expandable prosthetic valve that is delivered into the patient's body in a radially compressed configuration on a balloon of the balloon catheter 16 (as shown in FIG. 1) and then radially expanded to a radially expanded configuration at the deployment site by inflating the balloon (or by actuating another type of expansion device of the delivery apparatus). Further details regarding a plastically expandable heart valve that can be implanted using the devices disclosed herein are disclosed in U.S. Publication No. 2012/0123529, which is incorporated herein by reference. In other aspects, the prosthetic heart valve 12 can be a self-expandable heart valve that is restrained in a radially compressed configuration by a sheath or other component of the delivery apparatus and self-expands to a radially expanded configuration when released by the sheath or other component of the delivery apparatus. Further details regarding a self-expandable heart valve that can be implanted using the devices disclosed herein are disclosed in U.S. Publication No. 2012/0239142, which is incorporated herein by reference. In still other aspects, the prosthetic heart valve 12 can be a mechanically expandable heart valve that comprises a plurality of struts connected by hinges or pivot joints and is expandable from a radially compressed configuration to a radially expanded configuration by actuating an expansion mechanism that applies an expansion force to the prosthetic valve. Further details regarding a mechanically expandable heart valve that can be implanted using the devices disclosed herein are disclosed in U.S. Publication No. 2018/0153689, which is incorporated herein by reference. In still other aspects, a prosthetic valve can incorporate two or more of the above-described technologies. For example, a self-expandable heart valve can be used in combination with an expansion device to assist expansion of the prosthetic heart valve.

FIG. 2 illustrates an assembly (which can be referred to as an introducer device 90 or assembly) that can be used to introduce the delivery apparatus 10 and the prosthetic device (e.g., prosthetic heart valve 12) into a patient's body, according to one aspect. The introducer device 90 can comprise a housing 92 at a proximal end of the device and an expandable sheath 100 extending distally from the housing 92. The housing 92 can function as a handle for the device. Generally, during use, a distal end of the sheath 100 is passed through the skin of the patient and is inserted into a vessel, such as the femoral artery. The delivery apparatus 10 with its implant (e.g., prosthetic heart valve 12) can then be inserted through the housing 92 and the sheath 100, and advanced through the patient's vasculature to the treatment site, where the implant is to be delivered and implanted within the patient. In certain aspects, the introducer housing 92 can include a hemostasis valve that forms a seal around the outer surface of the guide catheter 14 once inserted through the housing to prevent leakage of pressurized blood.

In alternative aspects, the introducer device 90 need not include a housing 92. For example, the disclosed sheath 100 can be an integral part of a component of the delivery apparatus 10, such as the guide catheter. For example, the sheath can extend from the handle portion 18 of the guide catheter. Additional examples of introducer devices and expandable sheaths can be found in U.S. patent application Ser. No. 16/378,417, which is incorporated by reference in its entirety.

In still alternative aspects, the disclosed herein sheath 100 can be used with the introducer device 90 to deliver a stent or other implantable device. In still further aspects, the introducer device 90 having the disclosed sheath 100 can also be used to deliver non-self-expanding prostheses such as a balloon-expandable stent. Additional examples of introducer devices for the introduction of the stents are found in U.S. Pat. No. 6,344,044, which is incorporated by reference in its entirety.

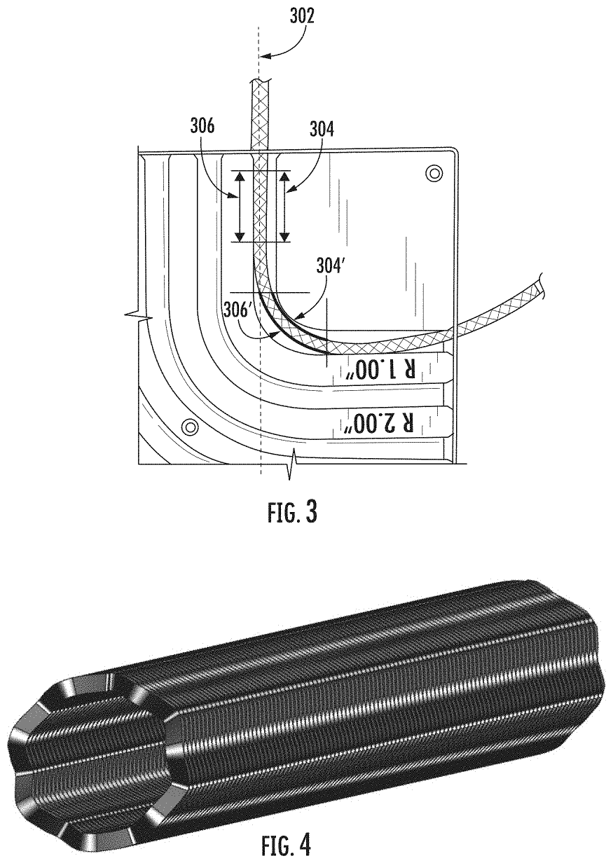

In still further aspects, disclosed herein is an expandable sheath for deploying a medical device comprising at least one plurality of struts arranged to form an expandable cylindrical frame having a proximal and distal end, an inner surface and an outer surface, and a central axis extending along a length of the frame; wherein the expandable cylindrical frame has a first length and an opposite second length, and wherein in a first position at least a portion of the first length and at least a portion of the second length are substantially the same; wherein the expandable cylindrical frame is movable from the first position to a second position by curving away from the central axis, wherein at the second position at least a portion of the first length shortens and at least a portion of the second length elongates; wherein the inner surface of the expandable cylindrical frame defines a lumen configured to receive a medical device; and wherein the expandable cylindrical frame is configured to expand from a first diameter d₁ in an unexpanded position to a second diameter d₂ an expanded position upon passage of a medical device. FIG. 3 shows an exemplary photograph of the disclosed sheath. The expandable sheath 300 shown in FIG. 3 comprises a frame and a polymeric material whose configurations are discussed in detail below. The expandable cylindrical frame has a central axis 302 and a first length and an opposite second length. As can be seen in FIG. 3, in a first position, at least a portion of the first length 304 and at least a portion of the second length 306 are substantially the same. It can be further seen that when the frame (and as a result, the sheath) moves from the first position to the second position by curving away from the central axis the at least a portion of the first length 304′ shortens, while at least a portion of the second length 306′ elongates. In still further aspects, the first length is provided on a first portion of an outer diameter of the expandable cylindrical frame, and the second length is provided on a second portion of the outer diameter of the expandable cylindrical frame, wherein the first portion is circumferentially opposite to the second portion around the outer diameter. Without wishing to be bound by any theory, it is hypothesized that in certain and unlimiting aspects, this configuration allows the frame (and as a result, the sheath) to be substantially kink-resistant when passing through the curved and folded pathways of the patient's vasculature.

In certain aspects, the first diameter d₁ of the frame in the unexpanded position can be from about 3 mm to about 5 mm, including exemplary values of about 3.2 mm, about 3.5 mm, about 3.7 mm, about 4 mm, about 4.2 mm, and about 4.8 mm. It is further understood that the first diameter d₁ can have any value between any two foregoing values, for example, and without limitation, the first diameter d₁ can be between 3 mm and 4 mm, or between 3.5 mm and 4.5 mm.

In yet other aspects, the second diameter d₂ of the frame in the expanded position can be from about 5 mm to about 10 mm, including exemplary values of about 5.2 mm, about 5.5 mm, about 5.7 mm, about 6 mm, about 6.2 mm, about 6.5 mm, about 6.7 mm, about 7 mm, 7.2 mm, about 7.5 mm, about 7.7 mm, about 8 mm, about 8.2 mm, about 8.5 mm, about 8.7 mm, about 9 mm, 9.2 mm, about 9.5 mm, and about 9.7 mm. It is further understood that the second diameter d₂ can have any value between any two foregoing values, for example, and without limitation, the second diameter d₂ can be between 5 mm and 6 mm, or between 5 mm and 8 mm.

In still further aspects, the frame disclosed herein is configured to contract to a diameter that is substantially the same as the first diameter d₁ upon removal and/or passage of the medical device. In such aspects, for example, and without limitation, when the first diameter d₁ expands upon passage of the medical device from about 4 mm to the second diameter d₂ of about 6 mm, the frame is configured to contract to a diameter substantially identical to about 4 mm. However, in some aspects, when expansion of the frame is larger due to the passage of the larger medical device, the return contraction to the first diameter can be not favorable, as it can result in hemorrhage and undesired risk to the patient. In such exemplary aspects, the frame can be configured to contract to a diameter that is from about 10% to about 90%, including exemplary values of about 15%, about 20%, about 25%, about 30%, about 35%, about 40%, about 45%, about 50%, about 55%, about 60%, about 65%, about 70%, about 75%, about 80%, and about 85% larger than the first diameter d₁ upon removal of the medical device. For example, and without limitation, when the first diameter d₁ expands upon passage of the medical device from about 4 mm to the second diameter d₂ of about 8 mm, the frame can be configured to contract to a diameter of about 6 mm, to prevent hemorrhage.

In still further aspects and as disclosed herein, the frame can have the first diameter d₁ that is substantially uniform along the length of the frame. However, in some exemplary aspects, the first diameter d₁ can vary along the length of the frame. In such exemplary aspects, the first diameter d₁ at the proximal end, where the frame (sheath) connects to the housing 92 (as shown in FIG. 2), can be larger than the first diameter d₁ at the distal end. In yet further aspects, the diameter d₁ can gradually decrease from the proximal end of the frame to the middle portion of the frame and then decrease again towards the distal end of the frame.

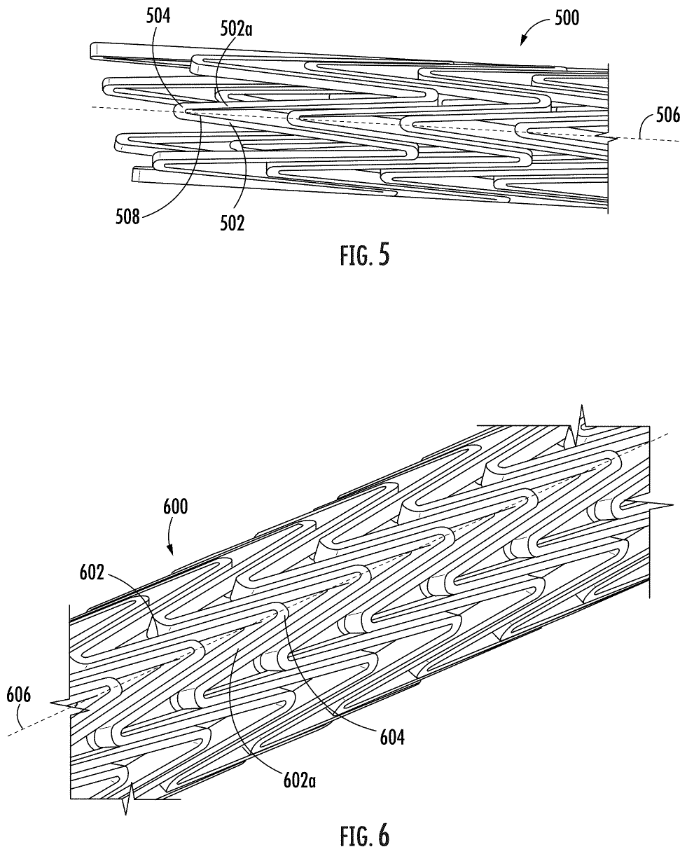

FIG. 4-7 show an exemplary frame, as disclosed in some aspects described herein. FIG. 4 shows an exemplary 3D schematic of the frame in the expanded configuration. FIGS. 5 and 6 show a more detailed configuration of the exemplary frame 500 at a distal end and the exemplary frame 600 in the middle of the sheath, respectively. In these exemplary aspects, the plurality of struts are arranged to form a zig-zag configuration comprising a plurality of substantially straight portions 502 and 502a or 602 and 602a circumferentially followed by a plurality of substantially bend portions 504 or 604 and wherein the zig-zag configuration is wound along the central axis to form the frame. In certain aspects, the zig-zag configuration is helically wound along the central axis to form the frame. It is understood that in certain aspects, the plurality of substantially straight portions (502, 502a, 602, 602a) can have an equal predetermined length. While in other aspects, the plurality of substantially straight portions (502, 502a, 602, 602a) can comprise one or more portions that can have unequal lengths, for example, at the distal and/or proximal ends of the frame to accommodate optional variations in diameter d₁ as described above. It is further understood that the length of each substantially straight portion of the plurality of substantially straight portions (502, 502a, 602, 602a) can be easily determined depending on the desired application and to what extent the frame is expected to expand. It can also be seen in FIG. 5 and FIG. 6 that the plurality of substantially bend portions (504, 604) can define a bisecting line 506, 606 extending through an apex of the plurality of substantially bend portions (504, 604) and extending longitudinally along a length of the sheath. In such aspects, each substantially straight portion of the plurality of substantially straight portions (502, 502a, 602, 602a) can form a first angle 508 with the bisecting line from 0° to about 90°, including exemplary angles of about 5°, about 10°, about 15°, about 20°, about 25°, about 30°, about 35°, about 40°, about 45°, about 50°, about 55°, about 60°, about 65°, about 70°, about 75°, about 80°, and about 85°.

In yet further aspects, it is understood that when the frame is in the expanded position, the first angle increases during the expansion by greater than 0% to about 1,000% of its original value, including exemplary value of about 10%, about 20%, about 30%, about 40%, about 50%, about 60%, about 70%, about 80%, about 90%, about 100%, about 150%, about 200%, about 250%, about 300%, about 350%, about 400%, about 450%, about 500%, about 550%, about 600%, about 650%, about 700%, about 750%, about 800%, about 850%, about 900%, and about 950%. In yet further aspects, it is understood that the first angle can be increased by any value between any two foregoing values, for example, the first angle can be increased by greater than 0% to about 600% of its original value, or by 10% to about 400%, or by 100% to about 800%.

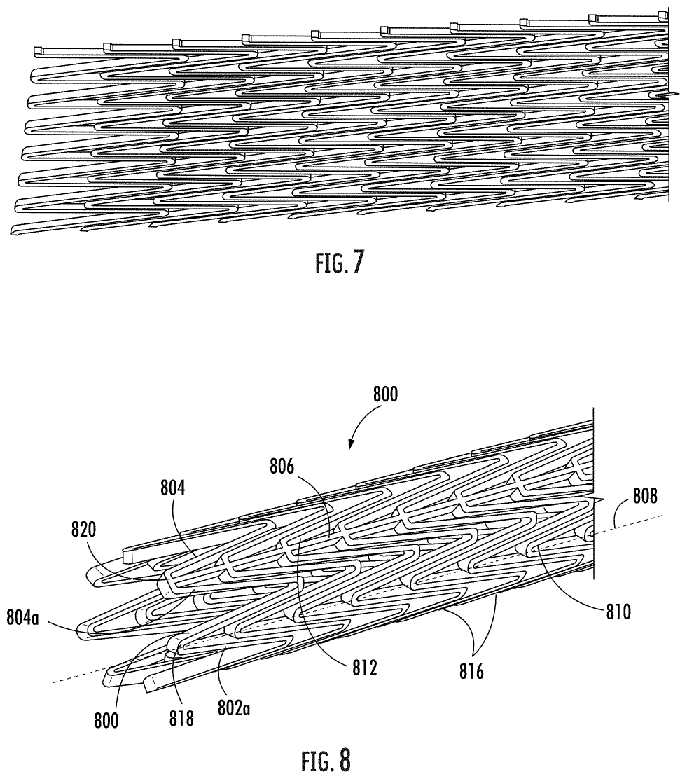

FIG. 7 shows a planar schematic of the frame configuration described in this aspect.

FIGS. 8-11 show an additional frame configuration according to other aspects of the disclosure. For example, FIGS. 8 and 9 show frame 800 and 900 configurations at the distal end and in the middle of the frame, respectively. In this exemplary aspect, the plurality of struts are arranged in a zig-zag configuration to form a plurality of parallel circumferential rows 816, wherein each row of the plurality of parallel circumferential rows 816 comprises a plurality of substantially straight portions 802 and 802a circumferentially followed by a plurality of substantially bend portions 818; and wherein the plurality of parallel circumferential rows 816 are axially coupled to each other by a singular vertical strut member 806 disposed along a length of the frame 800, 900. In some aspects, each substantially straight portion of the plurality of substantially straight portions 802, 802a can have equal lengths in this configuration; while in other aspects, the plurality of substantially straight portions 802, 802a can comprise one or more portions having unequal lengths, e.g., at the distal and/or proximal ends of the frame. Also, in some aspects, the plurality of substantially bend portions 818 can define a bisecting line 808 extending through an apex of the plurality of substantially bend portions 818 and extending longitudinally along a length of the sheath. In such aspects, each substantially straight portion of the plurality of substantially straight portions 802, 802a can form a first angle 810 with the bisecting line 808 from 0° to about 90°, including exemplary angles of about 5°, about 10°, about 15°, about 20°, about 25°, about 30°, about 35°, about 40°, about 45°, about 50°, about 55°, about 60°, about 65°, about 70°, about 75°, about 80°, and about 85°. In yet further aspects, it is understood that when the frame is in the expanded position, the first angle increases during the expansion by greater than 0% to about 1,000% of its original value, including exemplary value of about 10%, about 20%, about 30%, about 40%, about 50%, about 60%, about 70%, about 80%, about 90%, about 100%, about 150%, about 200%, about 250%, about 300%, about 350%, about 400%, about 450%, about 500%, about 550%, about 600%, about 650%, about 700%, about 750%, about 800%, about 850%, about 900%, and about 950%. In yet further aspects, it is understood that the first angle can be increased by any value between any two foregoing values, for example, the first angle can be increased by greater than 0% to about 600% of its original value, or by 10% to about 400%, or by 100% to about 800%.

In further aspects and as shown in FIG. 8, a singular vertical strut member 806 can extend longitudinally along a length of the sheath and connect apexes 820 of the substantially bending portions 818 of each row of the plurality of parallel circumferential rows 816. In such exemplary aspects, the singular vertical strut member 806 can form a second angle 812 with each substantially straight portion of the corresponding adjacent substantially straight portions 804, 804a of 0° to about 90°, including exemplary angles of about 5°, about 10°, about 15°, about 20°, about 25°, about 30°, about 35°, about 40°, about 45°, about 50°, about 55°, about 60°, about 65°, about 70°, about 75°, about 80°, and about 85°. In certain aspects, when the medical device is introduced into the frame as disclosed herein, the frame expands circumferentially in a direction opposite to the singular vertical strut member 806 while not expanding along the singular vertical strut member 806. In this exemplary aspect, the frame can have a “rod-like” behavior, able to substantially bend in any direction without kinking.

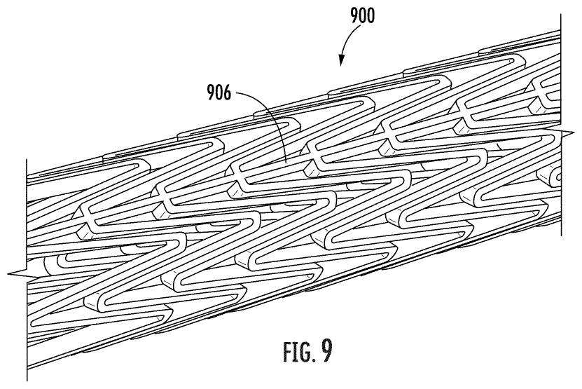

FIGS. 10-11 show a flattened view of various sections of the frame configuration described in the exemplary aspects having the singular vertical strut member 1006 and 1106, respectively.

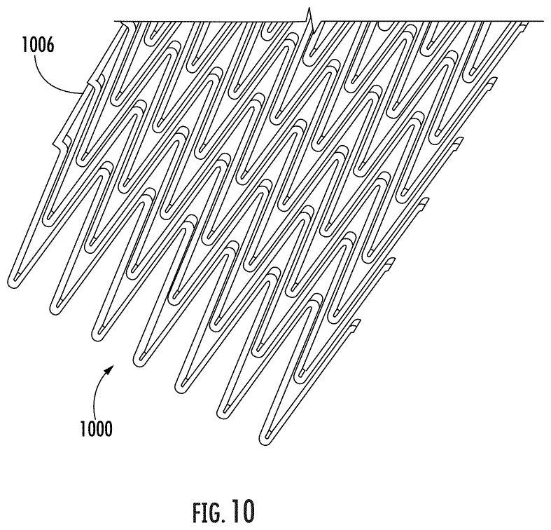

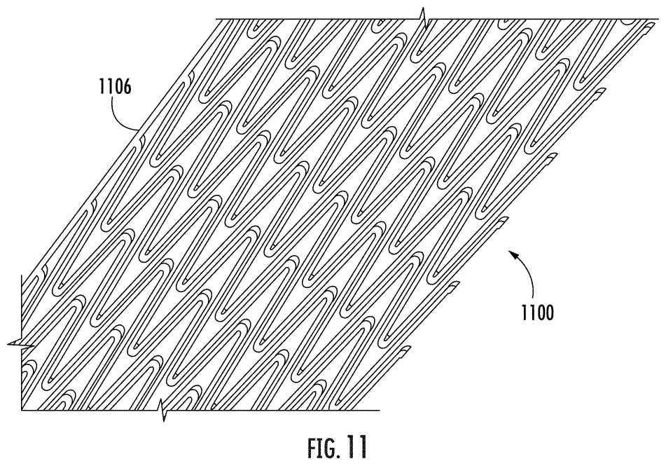

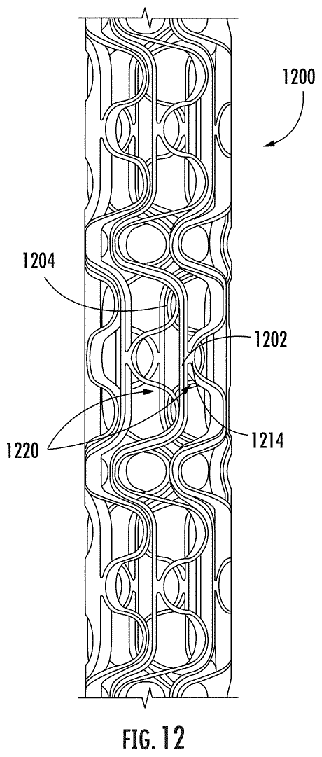

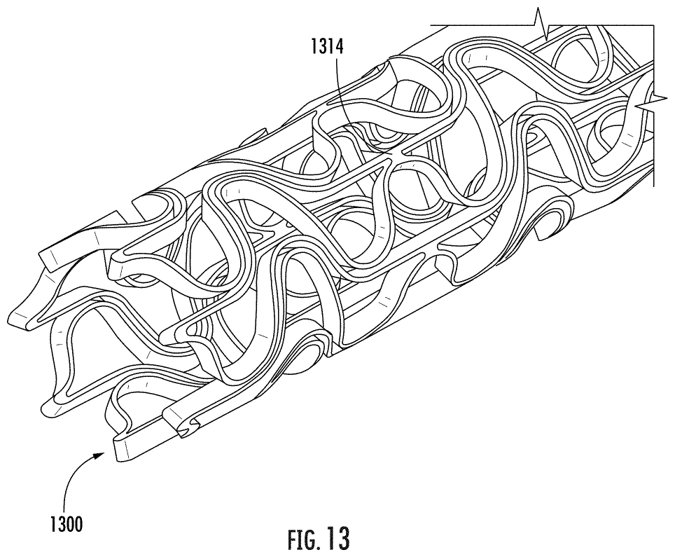

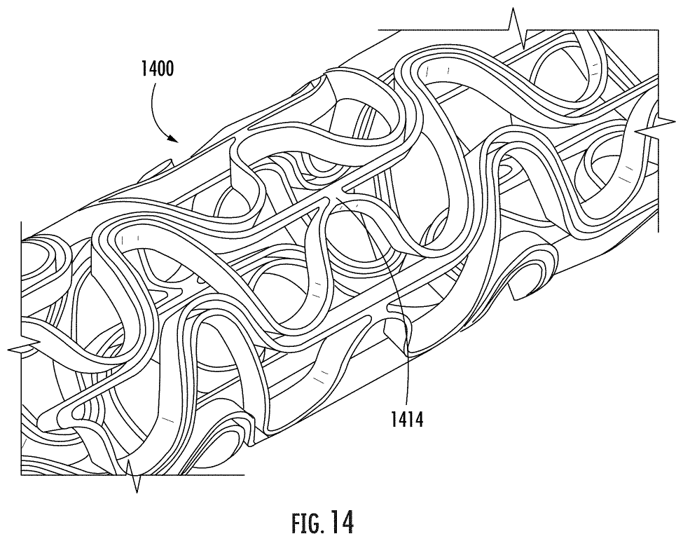

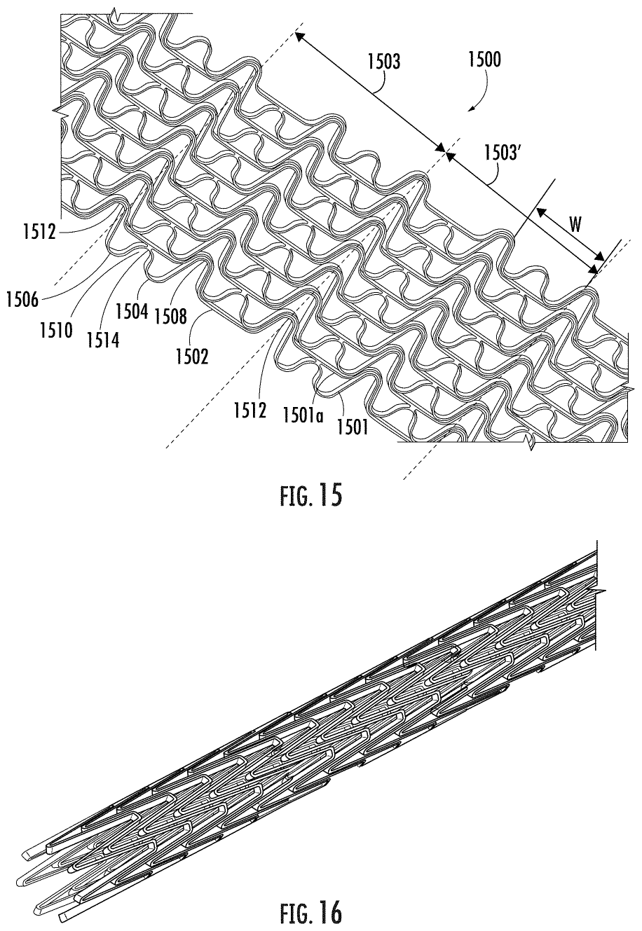

FIGS. 12-15 show an aspect where the plurality of struts are arranged in a configuration wherein each strut of the plurality of struts is able to individually elongate in a circumferential direction along the length of the frame upon introduction of the medical device. For example, frame 1200 shows a plurality of struts, where the plurality of struts are arranged along a length of the frame 1200 in a plurality of parallel axially extending rows 1220 each including corresponding first and second valleys 1202, 1204. An exemplary flattened schematic of the frame 1500 disclosed in these aspects is shown in FIG. 15. Each row of the plurality of parallel axial rows can comprise an undulating pattern unit 1503. In such exemplary aspects, each of the undulating pattern units 1503 can comprise a first valley 1502 having a substantially straight bottom portion, having a first width (w). The first valley, 1502, is then followed by a first apex 1508, a second valley 1504, a second apex 1510, and a third valley 1506. In such exemplary configuration, a total width of the second 1504 and third valleys 1506 and the second apex 1510 is substantially the same as the first width (w) of the substantially straight bottom portion of the first valley 1502.

In still further aspects, the third valley 1506 present in the frame 1500, as shown in FIG. 15, can be followed by a third apex 1512. In further aspects, the third apex 1512 can be followed by a first valley 1502 of a next undulating pattern unit in the same row. It is understood that in certain and unlimiting aspects, each of the valleys or apexes in the same undulating pattern unit 1503 and each subsequent undulating pattern unit 1503′ can be formed from the same material. While in other unlimiting aspects, at least a portion of each of the valleys or apexes in the same undulating pattern unit 1503 and each subsequent undulating pattern unit 1503′ be formed from different materials or a combination of materials known in the art and applicable for the desired applications. In certain aspects, each component follows other components continuously without the presence of any connections or coupling elements between each component. However, in other exemplary and unlimiting aspects, at least a portion of the components of each of the undulating pattern units 1503, 1503′ can be connected with a connector, or a coupling element, or a fastener. In such aspects, where the connectors, coupling elements, or fasteners are present, these elements can comprise any of such connectors, coupling elements, or fasteners known in the art and suitable for such purposed. Even further, it is understood that if any of those elements are present in the frame configuration, these elements can be made from any materials known in the art that can be used in the desired application.

In still further aspects, the plurality of parallel axial rows can be arranged such that a second apex 1510 in each repetitive undulating pattern unit 1503 of each row is positioned above and is coupled with at least a portion of a substantially straight bottom portion of a first valley 1502 of an adjacent row by a bridging member 1514. Bridging members 1214, 1314, and 1414 can also be seen in FIGS. 12-14. In still further aspects, the bridging member can be any member configured to connect or couple to elements of adjacent rows. In certain aspects, the bridging member 1214, 1314, 1414, 1514 can comprise a fastener. It is understood that in certain aspects, the fastener can comprise an adhesive material, such as, for example, a glue, or it can be a mechanical fastener, such as a weld, etc. It is understood that any materials that can be applicable for the desired application can be utilized as a fastener.

It is understood that each strut of the plurality of struts described in these aspects is configured to individually elongate. The configuration disclosed in these exemplary aspects allows the struts to move between elongated and shortened configurations at the longitudinal sections between bridging members.

It is also understood that in the disclosed configuration, the bridging member 1214, 1314, 1414, 1514 can assist in providing a desired expanded configuration of the frame.

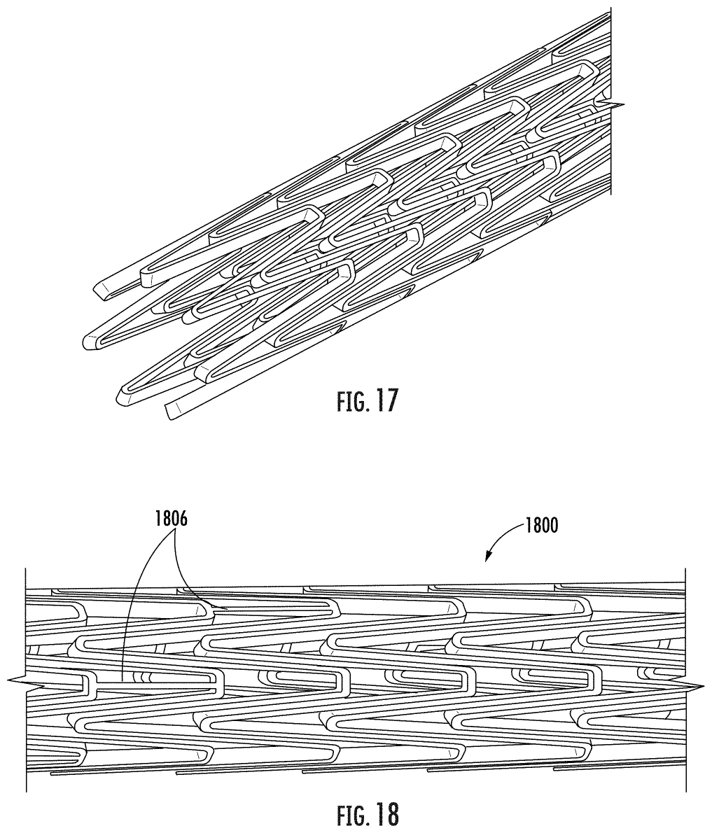

FIG. 16 shows an additional aspect of the disclosed frame, while FIGS. 17 and 18 show a magnified view of the distal end of the frame and the middle portion of the frame, respectively. FIG. 19 shows a flattened view of the frame in the same configuration. It can be seen that in this exemplary configuration, the plurality of struts are arranged in a zig-zag configuration to form a plurality of parallel circumferential rows 1920, wherein each row of the plurality of parallel circumferential rows 1920 comprises a plurality of straight portions circumferentially followed by a plurality of substantially bend portions; wherein adjacent rows of the plurality of parallel circumferential rows are coupled together by a singular bridging member (1806 or 1906) extending from a substantially bend portion in a first row 1920 to a corresponding substantially bend portion in an adjacent second row 1920. In such exemplary aspects, the bridging member 1806, 1906 coupling any two adjacent rows is circumferentially offset from a bridging member coupling any following two adjacent rows. The offset between the vertical bridging members 1806, 1906 can enable the frame to substantially bend in any direction without kinking. It is understood that the bridging member 1806, 1906 can be made from any material that is applicable for the desired application. It is further understood that the bridging member 1806, 1906 can be provided by any known in the art methods. For example, the bridging member 1806, 1906 can be added after the frame is formed by welding or glue. While in yet other aspects, the bridging member is formed simultaneously with the plurality of struts. In certain aspects and similarly to other zig-zag configurations, each substantially straight portion of the plurality of straight portions can have equal lengths. While in other aspects, the plurality of straight portions can comprise one or more portions having unequal lengths at the distal and/or proximal ends of the frame.

Also, in some aspects, similarly to other zig-zag configurations disclosed herein, the plurality of substantially bend portions disclosed in this aspect can define a bisecting line extending through an apex of each substantially bend portion of the plurality of substantially bend portions, the bisecting line extending longitudinally along a length of the sheath, and wherein each substantially straight portion of the plurality of substantially straight portions has a first angle from the bisecting line from 0° to about 90°, including exemplary angles of about 5°, about 10°, about 15°, about 20°, about 25°, about 30°, about 35°, about 40°, about 45°, about 50°, about 55°, about 60°, about 65°, about 70°, about 75°, about 80°, and about 85°. In yet further aspects, it is understood that when the frame is in the expanded position, the first angle increases during the expansion by greater than 0% to about 1,000% of its original value, including exemplary value of about 10%, about 20%, about 30%, about 40%, about 50%, about 60%, about 70%, about 80%, about 90%, about 100%, about 150%, about 200%, about 250%, about 300%, about 350%, about 400%, about 450%, about 500%, about 550%, about 600%, about 650%, about 700%, about 750%, about 800%, about 850%, about 900%, and about 950%. In yet further aspects, it is understood that the first angle can be increased by any value between any two foregoing values, for example, the first angle can be increased by greater than 0% to about 600% of its original value, or by 10% to about 400%, or by 100% to about 800%.

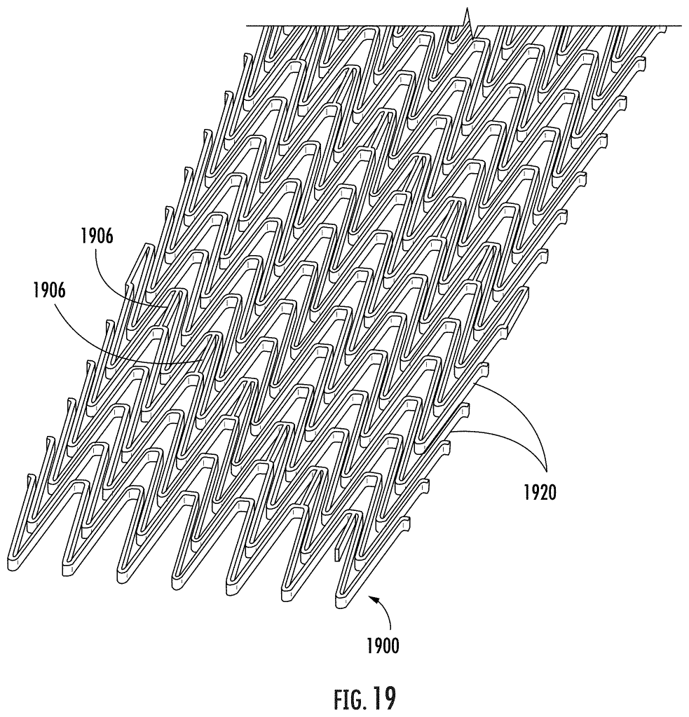

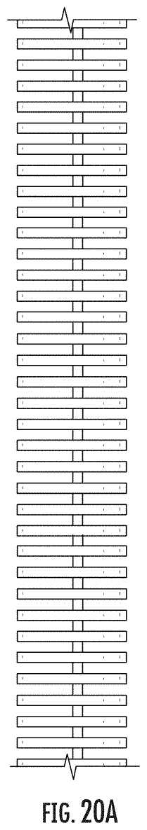

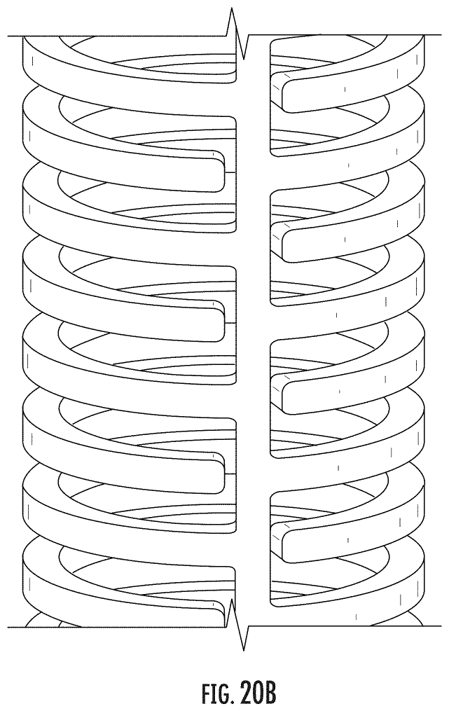

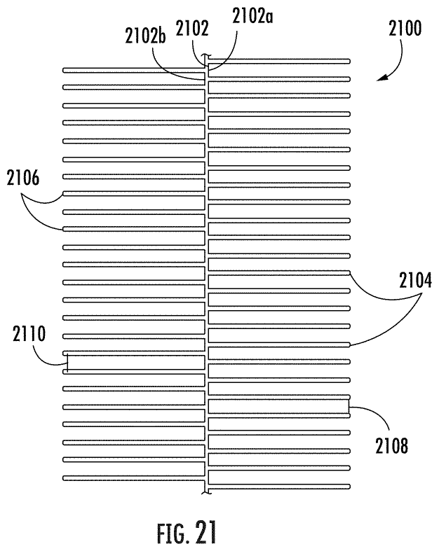

FIGS. 20-26 show a frame configuration in other aspects. FIG. 20A shows a front view of this exemplary frame, FIG. 20B shows a back perspective view of the frame 2100, and FIG. 21 shows a flattened view of the frame 2100. In this exemplary aspect, the cylindrical frame 2100 is formed by a first plurality of struts 2104 arranged along a first edge 2102a of a singular vertical member 2102 and a second plurality of struts 2106 arranged along an opposite second edge 2102b of the singular vertical member 2102. The first and second plurality of struts 2104, 2106 curve around the longitudinal axis of the frame forming a central lumen therethrough. In still further aspects, each strut of the first plurality of struts 2104 is spaced from each other such that a first gap 2108 is formed between two adjacent struts in the first plurality of struts 2104. While in other aspects, each strut of the second plurality of struts 2106 is spaced from each other such that a second gap 2110 is formed between two adjacent struts in the second plurality of struts 2106. As can be seen in FIG. 21, each strut of the first plurality of struts 2104 can be axially offset along the singular vertical member 2102 from each strut of the second plurality of struts 2106. It is understood that when this exemplary frame is present in the unexpanded position, each strut of the first plurality of struts 2104 can be at least partially disposed within the second gap 2110, and each strut of the second plurality of struts can be at least partially disposed within the first gap 2108.

It is understood that the vertical member can have a predetermined width. In such aspects, a width of the vertical member can be less than 25% of a circumference of the cylindrical frame in the unexpanded position, or less than 20% of a circumference of the cylindrical frame in the unexpanded position, or less than 15% of a circumference of the cylindrical frame in the unexpanded position, or less than 10% of a circumference of the cylindrical frame in the unexpanded position, or less than 5 of a circumference of the cylindrical frame in the unexpanded position. It is understood that one of ordinary skills in the art can determine the circumference of the cylindrical frame in the unexpanded position based on d₁ of the frame. In still further aspects, the width of the vertical member can be measured along a portion of the circumference of the cylindrical frame corresponding to the vertical member.

Also disclosed herein are aspects where a width of the vertical member is less than about 20% of a length of at least one strut of the first and second plurality of struts; less than about 15% of a length of at least one strut of the first and second plurality of struts; less than about 10% of a length of at least one strut of the first and second plurality of struts; or less than about 5% of a length of at least one strut of the first and second plurality of struts. In still further aspects, the length of at least one strut of the first and second plurality of struts is measured between a proximal end of the corresponding strut adjacent the vertical member, and a distal end of the corresponding strut.

Also disclosed are aspects where a width of each strut of the first and second plurality of struts is less than about 25% of a circumference of the cylindrical frame in the unexpanded position; or less than about 20% of a circumference of the cylindrical frame in the unexpanded position; less than about 15% of a circumference of the cylindrical frame in the unexpanded position; less than about 10% of a circumference of the cylindrical frame in the unexpanded position; less than about 5% of a circumference of the cylindrical frame in the unexpanded position. In yet further aspects, the width of each strut of the first and second plurality of struts is measured in an axial direction along a longitudinal axis of the cylindrical frame.

Also disclosed are the aspects where a width of each strut of the first and second plurality of struts corresponds to a width of the vertical member. In yet further aspects, a width of each strut of the first and second plurality of struts is less than a width of the vertical member. In still further aspects, a width of each strut of the first and second plurality of struts is greater than a width of the vertical member.

Also disclosed are the aspects where a thickness of each strut of the first and second plurality of struts corresponds to a thickness of the vertical member. In yet other aspects, wherein a thickness of each strut of the first and second plurality of struts is greater than a thickness of the vertical member. While still in other aspects, a thickness of each strut of the first and second plurality of struts is less than a thickness of the vertical member.

In still further aspects, the struts' width can be anywhere from about 0.1 mm to about 3 mm, including exemplary values of about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, 2.2 mm, about 2.3 mm, about 2.4 mm, about 2.5 mm, about 2.6 mm, about 2.7 mm, about 2.8 mm, and about 2.9 mm. It is understood that the struts can have any width value between any two foregoing values. For example and without limitation, the struts' width can be from about 0.2 mm to about 2.5 mm, or from about 0.5 mm to about 1.5 mm, or from about 1 mm to about 3 mm.

In still further aspects, the vertical member described herein can have the same width as the struts. However, in other aspects, the vertical member has a different width from the first plurality and the second plurality of the struts. In yet still further aspects, the vertical member can have a width anywhere from about 0.1 mm to about 3 mm, including exemplary values of about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, 2.2 mm, about 2.3 mm, about 2.4 mm, about 2.5 mm, about 2.6 mm, about 2.7 mm, about 2.8 mm, and about 2.9 mm. It is understood that the medical member can have any width value between any two foregoing values. For example and without limitation, the vertical member's width can be from about 0.2 mm to about 2.5 mm, or from about 0.5 mm to about 1.5 mm, or from about 1 mm to about 3 mm.

In still further aspects, the first and/the second gap can be anywhere from about 0.1 mm to about 3 mm, including exemplary values of about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, 2.2 mm, about 2.3 mm, about 2.4 mm, about 2.5 mm, about 2.6 mm, about 2.7 mm, about 2.8 mm, and about 2.9 mm. It is understood that the first and/or the second gap can have any value between any two foregoing values. For example, and without limitation, the first and the second gaps can be from about 0.2 mm to about 2.5 mm, or from about 0.5 mm to about 1.5 mm, or from about 1 mm to about 3 mm.

Also disclosed herein are the aspects where the thickness of the first and second plurality of struts is measured radially between in an inner surface and corresponding outer surface of each strut of the first and second plurality of struts, wherein the thickness of the vertical member is measured radially between an inner and outer surface of the vertical member. It is understood that any thickness can be used to make the struts, for example, from about 1 mil to about 200 mils, including exemplary values of about 2 mils, about 3 mils, about 5 mils, about 6 mils, about 7 mils, about 8 mils, about 9 mils, about 10 mils, about 20 mils, about 30 mils, about 40 mils, about 50 mils, about 60 mils, about 70 mils, about 90 mils, about 100 mils, about 120 mils, about 150 mils, and about 180 mils.

In yet further aspects, the thickness for the first and the second plurality of struts can be anywhere from about 0.1 mm to about 1.5 mm, including exemplary values of about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, and about 1.4 mm. It is understood that the thickness of the first plurality of struts and the second plurality of struts can have any value between any two foregoing values. It is also understood that any struts described in any of the disclosed herein aspects can have a thickness that falls in the disclosed above range.

In yet further aspects, the d₂ of the frame in the expanded position is between 5 mm and 10 mm, including exemplary values of about 5.5 mm, about 6 mm, about 6.5 mm, about 7 mm, about 7.5 mm, about 8 mm, about 8.5 mm, about 9 mm, and about 9.5 mm. In yet further aspects, the d₂ of the frame in the expanded position is between 5 mm and 9 mm.

Still further disclosed are the aspects where a width of the first gap and/or a width of the second gap is greater than a width of each strut of the first and second plurality of struts.

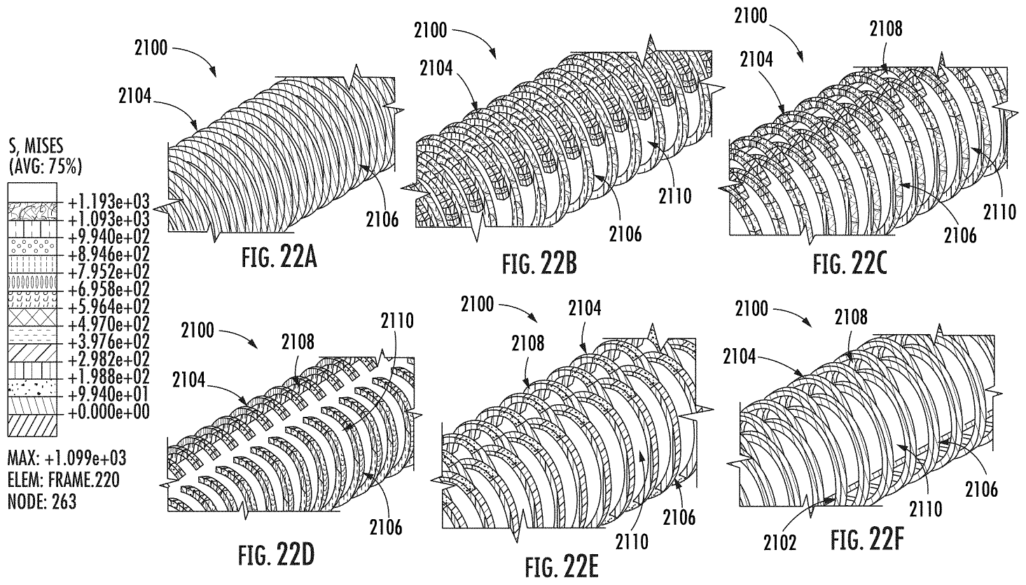

FIGS. 22-26 show simulation of the stress generated on the disclosed frame when it passes from the unexpanded to expanded position due to the passage of the medical device through the central lumen of the frame. FIGS. 22A-22F show a front perspective view of the frame as it expands from the unexpanded position FIG. 22A to maximum stress applied in the expanded position in FIG. 22D and return to the unexpanded position in FIG. 22E. As shown in FIG. 22A, when the frame in the unexpanded position, the frame experiences no stress. The stress associated with the passage of medical devices increases, as shown in FIGS. 22B-22D, with the greatest amount of stress is experienced along the middle length of the struts and the portion adjacent the vertical member 2102.

As shown in FIGS. 22B-22D, during expansion of the frame, the least amount of stress is experienced at the distal end of the struts (end opposite of the singular vertical member 2102). As shown in FIG. 22C, when the frame 2100 is fully expanded, the first and second plurality of struts 2104, 2106 are removed from their corresponding second and first gaps 2110, 2108, and a space/gap is provided between the distal ends of the struts 2104, 2106. That is, none of the first plurality of struts 2104 remains disposed within the second gap 2110, and none of the second plurality of struts 2106 remains disposed within the first gap 2108. In this configuration, the greatest amount of stress is experienced along the middle length of the strut and the portion adjacent the vertical member 2102, and the least amount of stress is experienced at the distal end of the struts. After the passage of a medical device, the frame contracts and the stress is reduced (FIGS. 22E, 22F). As discussed in detail above, the frame can contract to a diameter that is substantially the same as the first diameter d₁ upon removal of the medical device, or it can contract to a diameter that is from 10% to 90% larger than the first diameter d₁ upon removal of the medical device. As discussed in detail above, the degree of frame contraction upon removal of the medical device can be designed by one of ordinary skills in the art depending on the size of the medical device and the possibility of hemorrhage if the frame contracts too much. FIG. 22F shows an aspect where a dimeter to which the frame contracts after simulated removal of the medical device is slightly larger than the original diameter, as shown in FIG. 22A.

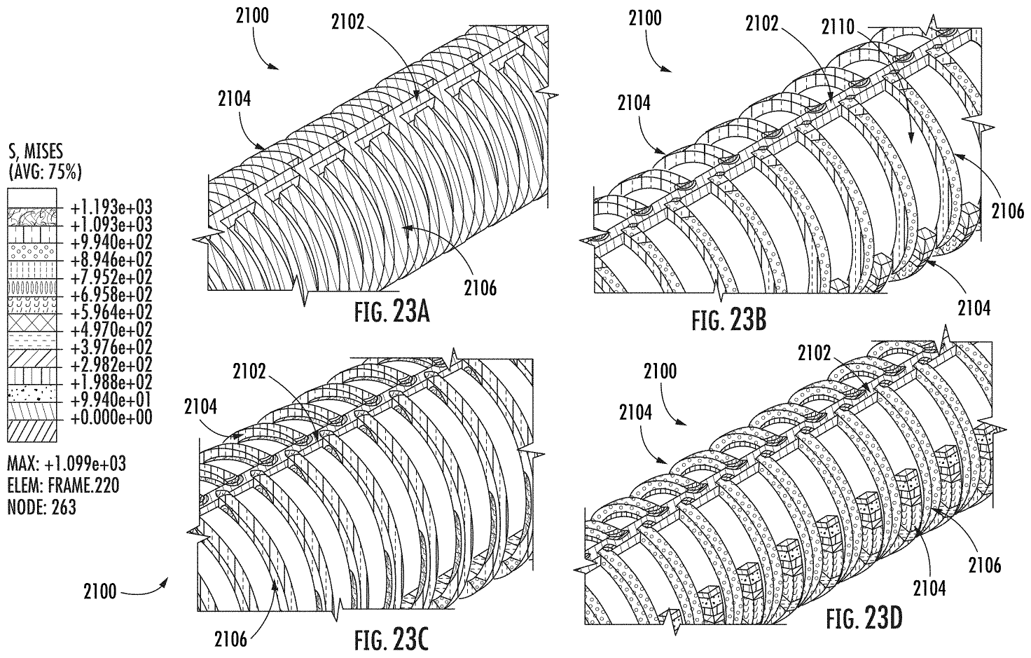

FIGS. 23-23D show a back perspective view of the frame as it expands from an unexpanded position FIG. 23A to the expanded position in FIG. 23C and returns towards an unexpanded position in FIG. 23D. As illustrated in FIG. 23A, the frame is in an unexpanded position and the least stress is experienced by the struts and the vertical member 2102. As the frame 2100 expands (FIGS. 23B-23C), increased stress is experienced along the first and second plurality of struts 2104, 2016. Increase stress is also experienced along the vertical member 2102 at the coupling between the vertical member 2102 and the struts 2104, 2106. As provided in FIGS. 23B-23D, the stress radiates out from the connection point between the struts 2104, 2106, and the vertical member 2102, with the most stress experienced adjacent the coupling point and reducing as it radiates outwards along the vertical member 2012. As shown in FIGS. 23B-23D, the stress at the coupling between the vertical member 2102 and the struts 2104, 2106 decreases as the struts return toward a non-expanded positioned (FIG. 23D).

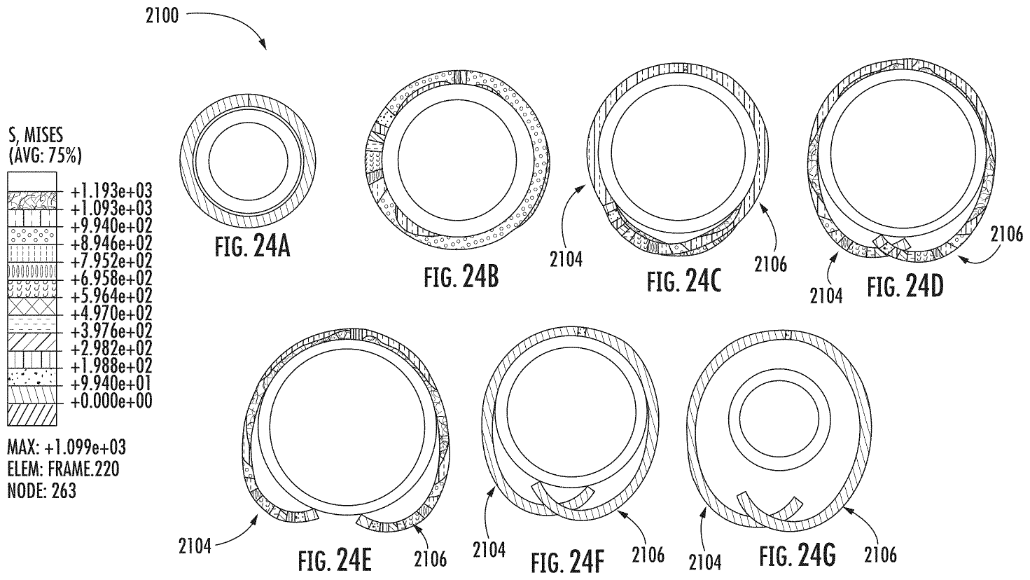

FIGS. 24A-24G show a cross-sectional/end view of the simulated stress results for the exemplary frame disclosed in FIGS. 20-21. It can be seen that in these exemplary aspects, as the frame 2100 expands (FIGS. 24B-24D), the first plurality of struts 2104 remains disposed in the second gap 2110, and the second plurality of struts 2106 remain disposed in the first gap 2108. When the frame 2100 is fully expanded (FIG. 24E), the struts 2104, 2106 are removed from their corresponding first and second gaps 2108, 2110, and a space is provided between the distal ends of the struts 2104, 2106. That is, none of the first plurality of struts 2104 remains disposed within the second gap 2110, and none of the second plurality of struts 2106 remains disposed within the first gap 2108. As illustrated in FIGS. 24B-24E, during expansion, the greatest amount of stress is experienced along the middle length of the struts and the portion adjacent the vertical member 2102, and the least amount of stress is experienced at the distal end of the struts (end opposite of the singular vertical member 2102). It can also be seen that the dimeter to which the frame 2100 contracts after simulated removal of the medical device is significantly larger (FIG. 24G) than the original diameter, as shown in FIG. 24A.

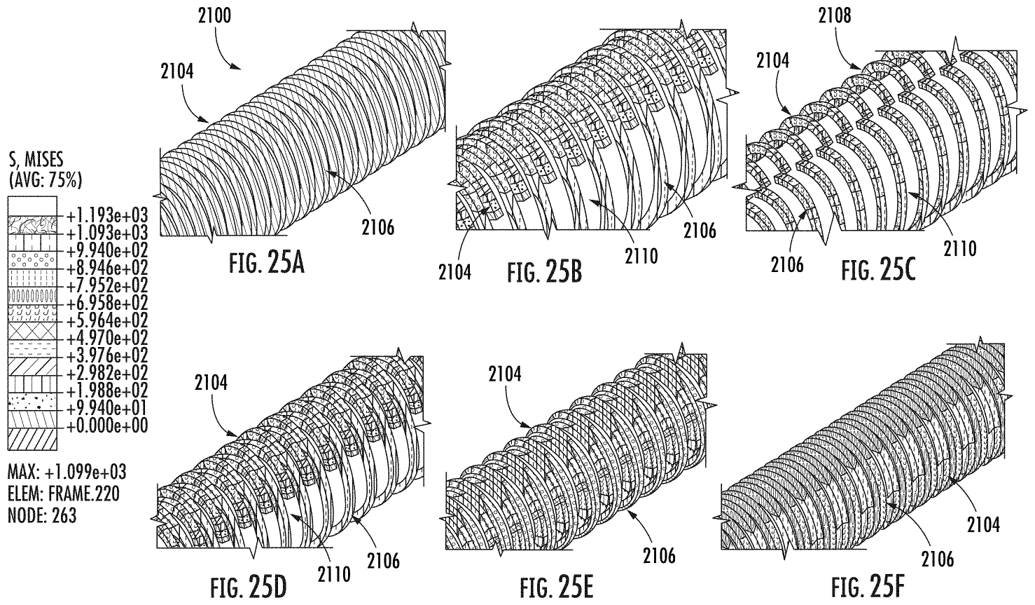

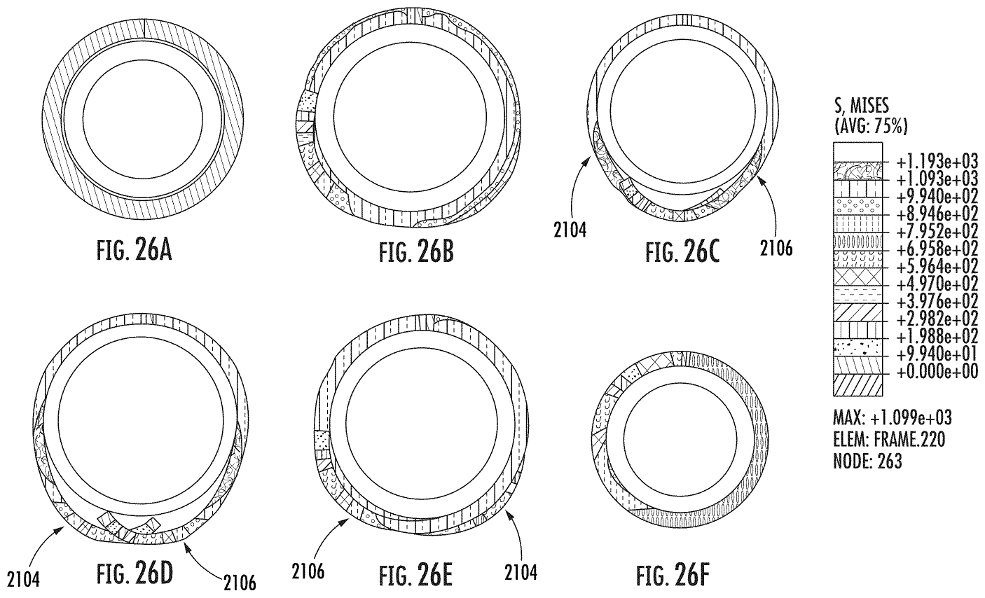

FIGS. 25-26 show side perspective and cross section/end views of the simulated results for the exemplary frame 2100 as disclosed in FIGS. 20-21. It can be seen that during the simulated passage of the medical device in this aspect when the frame 2100 is in the expanded position, at least a portion of the first plurality of struts 2104 remains at least partially disposed within the second gap 2110 and at least a portion of the second plurality of struts 2106 remains at least partially disposed within the first gap 2018 (FIGS. 25B-25E and FIGS. 26B-26E). That is, unlike the simulation in FIGS. 22-24, the first and second plurality of struts 2104, 2106 remain in the second and first gaps 2110, 2108, respectively, when the frame is fully expanded (FIG. 25C and FIG. 26D). It can be further seen that upon removal of the simulated medical device, the frame 2100 contracts to a diameter that is substantially the same as the frame's diameter in the unexpanded position (compare FIG. 25F and original unexpanded frame of FIG. 25A, see also FIG. 26A compared to FIG. 26G).

As shown in FIG. 25B and FIGS. 26B-C, as the frame 2100 is expanding, the greatest amount of stress is experienced along the middle length of the struts and the portion adjacent the vertical member 2102, and the least amount of stress is experienced at the distal end of the struts (the end opposite the vertical member 2102). Similarly, when the frame 2100 is fully expanded (FIGS. 25C, 26D), the greatest amount of stress is experienced along the middle length of the strut and the portion adjacent the vertical member 2102, and the least amount of stress is experienced at the distal end of the struts. As provided in FIGS. 25D-25E and FIG. 26E, a similar stress pattern is experienced as the frame 2100 returns to an unexpanded configuration. FIGS. 25F and 26G illustrate the frame 2100 returned to its final unexpanded configuration. As described above, upon removal of the medical device, the frame 2100 contracts to a diameter that is substantially the same as the frame's diameter in the unexpanded position. However, as illustrated in FIGS. 25F and 26G, the frame 2100 experiences slightly increased stress in the final unexpanded configuration than in the original unexpanded configuration (FIGS. 25A, 26A).

As described above, the plurality of struts can be of any suitable material, including, for example, stainless steel, cobalt-chrome alloys, polymer, or any combination thereof. In certain exemplary and unlimiting aspects, the plurality of struts can be made of a shape memory material such as a nickel-titanium alloy knows as Nitinol that allows the frame to expand and contract to substantially original diameter if needed. In yet other exemplary aspects, when the frame is designed to contract to a diameter of about 10% to about 90% larger than the original diameter, a ductile material, such as stainless steel, can be used. However, it is understood that also nitinol can be used in the aspects where the frame is designed to contract to a diameter of about 10% to about 90% larger than the original diameter. In yet further aspects, the plurality of struts can comprise a nitinol, stainless steel, cobalt-chrome alloys, polymer, or any combination thereof. It is understood that if the polymer is chosen as a material for a plurality of struts, such polymer is a medical-grade polymer having the desired elastic properties. In yet further aspects, at least a portion of the disclosed herein frame exhibits an elastic deformation.

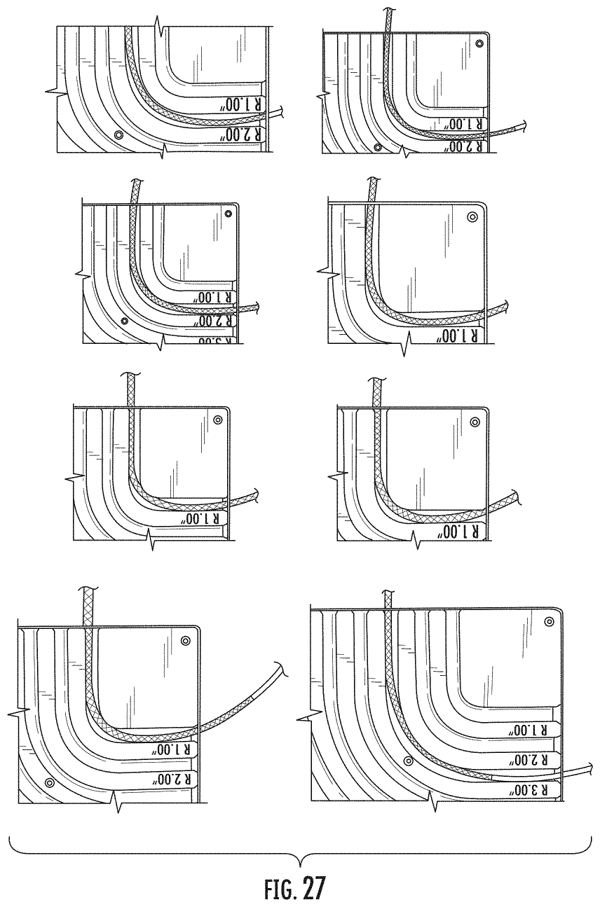

FIG. 27 shows a photograph of an exemplary sheath comprising the disclosed herein expandable cylindrical frame as it curves while it passes through various conduits while maintaining kink resistance. It can be seen that the expandable cylindrical frame as it passes through a curved conduit can bend to form a curved portion having a radius corresponding to the curvature of the conduit. For example, the cylindrical frame can form a curve having a radius from about 1 inch to about 3 inches, including exemplary values of about 1.2 inches, about 1.5 inches, about 1.7 inches, about 2 inches, about 2.2 inches, about 2.5 inches, and about 2.7 inches without exhibiting any kinks.

In still further aspects, the sheath disclosed herein exhibits a decrease in an insertion force by greater than 0% to about 100%, including exemplary values of about 5%, about 10%, about 20%, about 30%, about 40%, about 50%, about 60%, about 70%, about 80%, and about 90%. It is understood that the decrease in the insertion force can have any percentage value between any two foregoing values.

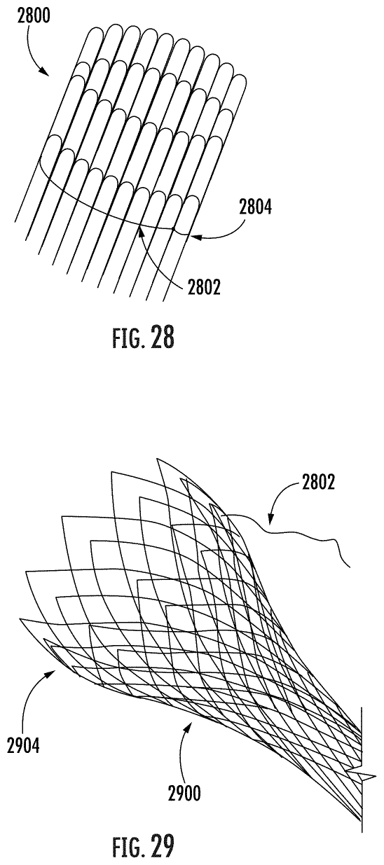

In still further aspects, the distal end of the frame 2800 can be modified to further facilitate the passage of the medical device (FIGS. 28 and 29). In such aspects, the distal end of the frame 2800 can comprise a pre-shaped flare (FIG. 29) that is configured to stay in a compressed state at the unexpanded position and to assume a flared configuration upon passing the medical device. It is understood that in such aspects, the pre-shaped flare can be kept in a compressed state by any means. In certain exemplary aspects, the pre-shaped flare can be kept in the compressed state by a filament 2802 attached to one or more of the plurality of struts 2804 at the distal end of the frame 2800. The filament 2802 can extend circumferentially around the plurality of struts 2804 at the distal end 2900 of the frame 2800. For example, the filament 2802 can extend circumferentially around all or a portion of the circumference of the distal end 2900 of the frame 2800. It is understood that the term “filament” as referred in this aspect is not limiting to one filament and can, in fact, comprise a plurality of filaments, a string, or a suture.

It is further understood that the filament can be removably or fixedly attached to one or more of the plurality of struts (2804, FIG. 28) by any methods known in the art. In certain aspects, it can be sutured to one or more struts. While in other aspects, it can be glued or tied to one or more struts. In certain aspects, the filament 2802 can be attached to any portion of the struts of any frame configuration disclosed herein. In yet further aspects, when the medical device passes through the distal end 2900 of the sheath, as shown in FIG. 29, the filament 2802 is configured to tear away from the frame 2800 while still comprising at least one point of attachment with the one or more struts 2804, thereby allowing the distal end 2900 of the frame 2800 to assume a flared configuration 2904 and increase the diameter of the opening at the distal end of the sheath. It is understood that maintaining at least one point of attachment with the struts 2804 prevents the filament 2802 from being released to the bloodstream upon tearing thereof. It is understood that in such aspects, the distal end 2900 can be heat set to flare or expand radially outwardly. In yet further aspects, the outward flare at the distal end 2900 can be provided with sufficient flexibility that the force exerted thereby on the surrounding arterial wall does not damage the tissue.

In still further aspects and as described here, the sheath can comprise one or more polymeric layers. In certain aspects, the one or more polymeric layers can comprise one or more of polyethylene, polypropylene, polyether block amide (PEBAX®), silicon, or any combination thereof. In yet further aspects, at least a portion of the frame is embedded within the one or more polymeric layers. It is understood that the polymeric layers can comprise two or more layers. In certain aspects, the frame can be positioned between two or more polymeric layers. In the aspects where two or more polymeric layers are present, these polymeric layers can comprise the same or different material.

In certain aspects, a polymeric layer can be positioned such that it surrounds the frame as one cylindrical layer. It is understood that in such an aspect, at least a portion of the frame can be embedded within the layer. While in other aspects, the polymeric layer can be adjacent to an outer surface of the frame without at least a portion of the frame being embedded within it. In still further aspects, the sheath can comprise at least two polymeric layers that are positioned such that one polymeric layer is adjacent to an outer surface of the frame, and another polymeric layer is adjacent to an inner surface of the frame. In certain aspects, the frame can be at partially embedded in one of those polymeric layers. While in other aspects, the frame can be at least partially embedded in two of those exemplary polymeric layers. In yet still further aspects, the disclosed two exemplary polymeric layers can be in at least partial contact with each other.

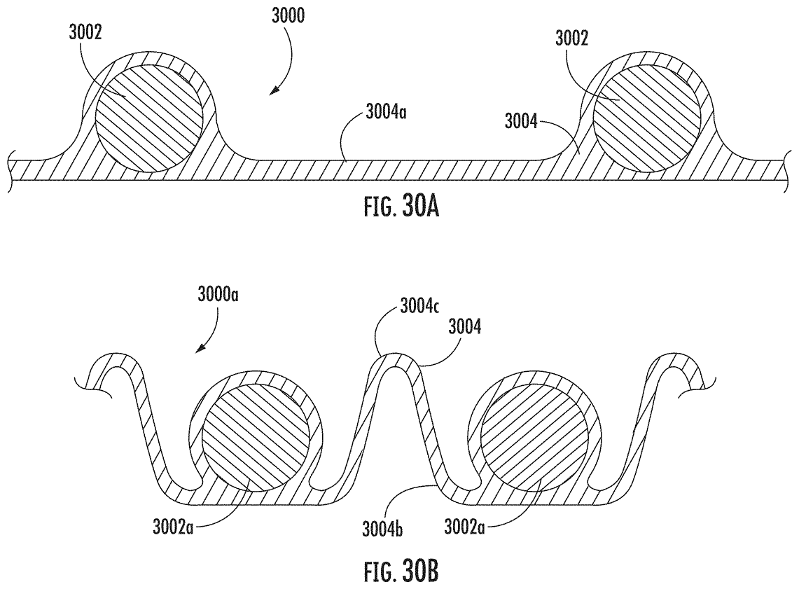

In still further aspects, the one or more polymeric layers can include a plurality of longitudinally-extending folds that extend circumferentially around the diameter of the sheath when the sheath is at the first diameter d₁. The longitudinally extending folds create a plurality of circumferentially spaced ridges and a plurality of circumferentially spaced valleys. As a medical device is passed through the sheath, the ridges and valleys level out to allow the sheath to radially expand. In yet further aspects, the one or more polymeric layers have a first thickness wherein the sheath at the first diameter d₁. In such exemplary aspects, as the medical device passes through the sheath, the one or more polymeric layers are thinning out to a second thickness to allow the sheath to radially expand. An exemplary aspect is shown in FIGS. 30A-30B. FIG. 30A illustrates a cross-sectional view of the sheath 3000a along its longitudinal axis. The sheath can have any of the frame configurations disclosed herein. For example, the sheath 3000a can have an exemplary frame configuration similar to those shown in FIGS. 20-26. FIG. 30A shows the exemplary rows 3002a of the frame in the unexpanded position and surrounded by an exemplary polymeric layer 3004. In the unexpanded position (FIG. 30A), the polymeric layer has a plurality of circumferentially spaced ridges 3004c and a plurality of circumferentially spaced valleys 3004b positioned between each row of the struts of the frame. When sheath 3000 expands (FIG. 30B), the ridges and valleys level out to form an extended polymeric layer 3004a and 3000 around the embedded rows 3002 of the frame. When the sheath is back to the unexpanded position (FIG. 30A), the ridges and valleys are formed again.

In still further aspects, the plurality of struts of the disclosed frame configurations can be movable between one or more polymeric layers such that the plurality of struts can radially expand as a medical device is passed through the sheath while the length of the sheath remains substantially constant, and, in some aspects, the plurality of struts are not engaged or adhered to one or more polymeric layers at all.

In certain aspects, the one or more polymeric layers can comprise a relatively thin layer of polymeric material. For example, in some aspects, the thickness of the one or more polymeric layers can be from 0.01 mm to 0.5 mm, including exemplary values of about 0.05 mm, about 0.1 mm, about 0.15 mm, about 0.2 mm, about 0.25 mm, about 0.3 mm, about 0.35 mm, about 0.4 mm, and about 0.45 mm.

In certain examples, the one or more polymeric layers can comprise a lubricious, low-friction, and/or relatively non-elastic material. In particular aspects, the one or more elastomeric materials can comprise a polymeric material having a modulus of elasticity of 400 MPa or greater. Exemplary materials can include ultra-high-molecular-weight polyethylene (UHMWPE) (e.g., Dyneema®), high-molecular-weight polyethylene (HMWPE), or polyether ether ketone (PEEK). In still further aspects, the materials can also comprise low-density polyethylene (LDPE), bi-oriented polypropylene, cast polypropylene, thermoplastic polyurethane, and/or combinations of any of the above. It is understood that such a low coefficient of friction materials can facilitate passage of the prosthetic device through the lumen of the sheath. Other suitable materials for the one or more polymeric layers can include polytetrafluoroethylene (PTFE), expanded polytetrafluoroethylene (ePTFE), ethylene tetrafluoroethylene (ETFE), nylon, polyethylene, polyether block amide (e.g., Pebax), and/or combinations of any of the above. Some aspects of the disclosed herein sheath can include a lubricious liner on an inner surface of the one or more polymeric layers surrounding the frame. Examples of suitable lubricious liners include materials that can further reduce the coefficient of friction, such as PTFE, polyethylene (such as, for example, HMWPE, UHMWPE, LDPE, HDPE), polyvinylidine fluoride, and combinations thereof. Suitable materials for a lubricious liner also include other materials desirably having a coefficient of friction of 0.1 or less.

In yet further aspects, the sheath disclosed herein can comprise an additional outer layer. In such exemplary aspects, the outer layer can be adjacent to one or more polymeric layers surrounding the frame. In such aspects, the outer layer can be an additional resilient, elastic layer. In certain aspects, the outer layer can be configured to apply force to the one or more polymeric layers in a radial direction when the sheath expands beyond its natural diameter by passage of the delivery apparatus through the sheath. Stated differently, the outer layer can be configured to apply encircling pressure to one or more polymeric layers, and the frame beneath the outer layer counteracts expansion of the sheath. The radially inwardly directed force is sufficient to cause the sheath to collapse radially back to its unexpanded state after the delivery apparatus is passed through the sheath. In still further aspects, the outer layer, as disclosed herein, can also take other forms, such as a tubular layer comprising an elastomeric material, a mesh, a shrinkable polymer layer such as a heat-shrink tubing layer, etc. In lieu of, or in addition to, the outer layer, the disclosed herein sheath can also include an elastomeric or heat-shrink tubing layer around the one or more polymeric layers. Examples of such elastomeric layers are disclosed in U.S. Publication No. 2014/0379067, U.S. Publication No. 2016/0296730, and U.S. Publication No. 2018/0008407, which are incorporated herein by reference.

In still further aspects, the disclosed herein sheath can comprise an exterior hydrophilic coating on an outer surface of the one or more polymeric layers. Such a hydrophilic coating can facilitate the insertion of the sheath into a patient's vessel, reducing potential damage. Examples of suitable hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N.V, Heerlen, the Netherlands), as well as other hydrophilic coatings (e.g., PTFE, polyethylene, polyvinylidene fluoride). Such hydrophilic coatings may also be included on an inner surface of the one or more polymeric layers to reduce friction between the sheath and the delivery system, thereby facilitating the use and improving safety. In some aspects, a hydrophobic coating, such as Perylene, can be used in order to reduce friction.

Various examples of polymeric layers and their configuration in the sheath can be found in U.S. Patent Application No. 62,912,569, Ser. Nos. 16/378,417, and 15/875,706 that are incorporated herein in their whole entirety.

### Methods

Turning out to the methods of making the expandable sheath disclosed herein. In certain aspects disclosed herein is a method comprising arranging at least one plurality of struts to form an expandable cylindrical frame having a proximal and distal end, an inner surface and an outer surface, and a central axis extending along a length of the frame; wherein the expandable cylindrical frame has a first length and an opposite second length, and wherein in a first position at least a portion of the first length and at least a portion of the second length are substantially the same; wherein the expandable cylindrical frame is movable from the first position to a second position by curving away from the central axis, wherein at the second position at least a portion of the first length shortens and at least a portion of the second length elongates; wherein the inner surface of the expandable cylindrical frame defines a lumen configured to receive a medical device; and wherein the expandable cylindrical frame is configured to expand from a first diameter d₁ in an unexpanded position to a second diameter d₂ an expanded position upon passage of a medical device. It is understood that the methods disclosed herein include arranging that the plurality of struts such that any of the disclosed herein expandable cylindrical configurations can be formed.

It is further understood that the plurality of struts can be formed within the specific configuration by laser cutting desired patterns and arranging them in the desired configurations. In yet other aspects, the plurality of struts and configurations can be formed by any other known in the art methods. For example, and without limitation, such configurations can be formed by 3-D printing, molding, etc.

In still further aspects, the methods disclosed herein comprise forming a pre-shaped flare at the distal end of the frame such that the pre-shaped flare is configured to stay compressed at the rest position and to assume a flared configuration upon passing the medical device. In such exemplary aspects, the pre-shaped flare can be prepared by a heat-set of the distal portion of the plurality of the struts. In yet further aspects, the filament can be attached to the distal portion of the plurality of struts by any methods known in the art. For example, and without limitations, the filament can be attached to the plurality of struts by methods comprising gluing, tying, welding, suturing, or any combination thereof.

In yet further aspects, the methods disclosed herein comprise applying one or more polymeric layers to the expandable cylindric frame. It is understood that such layers can be applied by any methods known in the art. In some aspects, the frame can be sandwiched between two or more polymeric layers. Yet, in other aspects, the frame can be inserted in a cylindrical tubing comprising one or more disclosed herein polymeric layers. In yet further aspects, the frame can be dipped into a solution comprising the desired polymer to form the one or more polymeric layers. Yet, in still further aspects, the polymeric layers can be formed by wrapping at least one layer of the polymeric film around the frame. In yet still further aspects, two or more layers of the polymeric films can be wrapped around the frame. In yet still, further aspects, if the two or more layers of the polymeric film are present, these layers can comprise the same polymer or different polymers. In such exemplary and unlimiting aspects, a first polymer can be wrapped around the frame to form one or more layers, and then a second polymer can be outwardly positioned on the first polymer by wrapping or dipping into the polymer solution or by positioning a tube comprising the second polymer, and so on, and then reflowing all layers together to form a laminate material.

In some aspects, where application of heat and pressure is needed during the method of making the expandable sheath, it can be achieved by placing the mandrel in a vessel containing a thermally-expandable material and heating the thermally-expandable material in the vessel. In some aspects, a radial pressure of 100 MPa or more is applied to the mandrel via the thermally-expandable material. In some aspects, applying heat and pressure further comprises applying a heat shrink tubing layer over one or more polymeric layers and applying heat to the heat shrink tubing layer. Additionally, exemplary methods of forming the one or more layers can be found in U.S. patent application Ser. No. 16/378,417, the content of which is incorporated herein in its whole entirety.

### EXEMPLARY ASPECTS

EXAMPLE 1: An expandable sheath for deploying a medical device comprising: at least one plurality of struts arranged to form an expandable cylindrical frame having a proximal and distal end, an inner surface and an outer surface, and a central axis extending along a length of the frame; wherein the expandable cylindrical frame has a first length and an opposite second length, and wherein, in a first position, at least a portion of the first length and at least a portion of the second length are substantially the same, wherein the expandable cylindrical frame is movable from the first position to a second position by curving away from the central axis, wherein at the second position at least a portion of the first length shortens and at least a portion of the second length elongates; wherein the inner surface of the expandable cylindrical frame defines a lumen configured to receive a medical device; and wherein the expandable cylindrical frame is configured to expand from a first diameter d₁ in an unexpanded position to a second diameter d₂ in an expanded position upon passage of a medical device.

EXAMPLE 2: The expandable sheath of any examples herein, particularly example 1, wherein the portion of the first length and the portion of the second length in the second position are located along a curved portion of the frame.

EXAMPLE 3: The expandable sheath of any examples herein, particularly examples 1 or 2, wherein the first length is provided on a first portion of an outer diameter of the expandable cylindrical frame and the second length is provided on a second portion of the outer diameter of the expandable cylindrical frame, wherein the first portion is circumferentially opposite to the second portion of the outer diameter.

EXAMPLE 4: The expandable sheath of any examples herein, particularly examples 1-3, wherein the expandable cylindrical frame is kink-resistant

EXAMPLE 5: The expandable sheath of any examples herein, particularly examples 1-4, wherein the first diameter d₁ is from about 3 mm to about 5 mm.

EXAMPLE 6: The expandable sheath of any examples herein, particularly examples 1-5, wherein the second diameter d₂ is from about 6 to about 10 mm.

EXAMPLE 7: The expandable sheath of any examples herein, particularly examples 1-6, wherein the plurality of struts are arranged to form a zig-zag configuration comprising a plurality of substantially straight portions circumferentially followed by a plurality of substantially bend portions and wherein the zig-zag configuration is wound along a central axis to form the frame.

EXAMPLE 8: The expandable sheath of any examples herein, particularly example 7, wherein each substantially straight portion of the plurality of substantially straight portions being of equal lengths.

EXAMPLE 9: The expandable sheath of any examples herein, particularly examples 7 or 8, wherein the plurality of substantially straight portions comprise one or more portions having unequal lengths at the distal and/or proximal ends of the frame.

EXAMPLE 10: The expandable sheath of any examples herein, particularly examples 7-9, wherein the plurality of substantially bend portions define a bisecting line extending through an apex of the plurality of substantially bend portions and extending longitudinally along a length of the sheath, and wherein each substantially straight portion of the plurality of substantially straight portions has a first angle from the bisecting line from 0° to about 90°.

EXAMPLE 11: The expandable sheath of any examples herein, particularly example 10, wherein when the frame is in the expanded position, the first angle increases by from greater than 0% to about 600% of its original value.

EXAMPLE 12: The expandable sheath of any examples herein, particularly examples 1-6, wherein the plurality of struts are arranged in a zig-zag configuration to form a plurality of parallel circumferential rows, wherein each row of the plurality of parallel circumferential rows comprises a plurality of substantially straight portions circumferentially followed by a plurality of substantially bend portions; and wherein the plurality of parallel circumferential rows are axially coupled to each other by a singular vertical strut member disposed along a length of the frame.

EXAMPLE 13: The expandable sheath of any examples herein, particularly example 12, wherein each substantially straight portion of the plurality of substantially straight portions being of equal lengths.

EXAMPLE 14: The expandable sheath of any examples herein, particularly examples 12 or 13, wherein the plurality of substantially straight portions comprise one or more portions having unequal lengths at the distal and/or proximal ends of the frame.

EXAMPLE 15: The expandable sheath of any examples herein, particularly examples 12-14, wherein the plurality of substantially bend portions define a bisecting line extending through an apex of the plurality of substantially bend portions and extending longitudinally along a length of the sheath, and wherein each substantially straight portion of the plurality of substantially straight portions has a first angle from the bisecting line 0° to about 90°.

EXAMPLE 16: The expandable sheath of any examples herein, particularly example 15, wherein when the frame is in the expanded position, the first angle increases by greater than 0% to about 600% of its original value.

EXAMPLE 17: The expandable sheath of any examples herein, particularly examples 12-16, wherein the singular vertical strut member extends longitudinally along a length of the sheath and connects apexes of the substantially bend portions of each row of the plurality of parallel circumferential rows, and the vertical strut member forms a second angle with each of the corresponding adjacent substantially straight portions of 0° to about 90°.

EXAMPLE 18: The expandable sheath of any examples herein, particularly examples 12-17, wherein upon introduction of the medical device, the frame expands circumferentially in a direction opposite to the single vertical strut member.

EXAMPLE 19: The expandable sheath of any examples herein, particularly examples 12-18, wherein upon introduction of the medical device, the frame does not expand along the singular vertical strut member.

EXAMPLE 20: The expandable sheath of any examples herein, particularly examples 1-6, wherein the plurality of struts are arranged in a configuration wherein each strut of the plurality of struts is able to individually elongate in a circumferential direction along the length of the frame upon introduction of the medical device.

EXAMPLE 21: The expandable sheath of any examples herein, particularly example 20, wherein the plurality of struts are arranged in a plurality of parallel axial rows, and wherein each row of the plurality of parallel axial rows comprises an undulating pattern unit, wherein each unit comprises: a first valley having a substantially straight bottom portion, wherein the substantially straight bottom portion of the first valley has a first width, wherein the first valley is followed by a first apex, a second valley, a second apex, and a third valley, and wherein a total width of the second and third valleys and the second apex is substantially the same as the first width of the substantially straight bottom portion of the first valley.

EXAMPLE 22: The expandable sheath of any examples herein, particularly example 21, wherein the third valley is followed by a third apex and a first valley of a next undulating pattern unit in the same row.

EXAMPLE 23: The expandable sheath of any examples herein, particularly example 21 or 22, wherein the plurality of parallel axial rows are arranged such that a second apex in each repetitive pattern unit of each row is positioned above and is coupled with at least a portion of a substantially straight bottom portion of a first valley of an adjacent row by a bridging member.

EXAMPLE 24: The expandable sheath of any examples herein, particularly examples 21-23, wherein the bridging member comprises a fastener.

EXAMPLE 25: The expandable sheath of any examples herein, particularly examples 1-6, wherein the plurality of struts are arranged in a zig-zag configuration to form a plurality of parallel circumferential rows, wherein each row of the plurality of parallel circumferential rows comprises a plurality of substantially straight portions circumferentially followed by a plurality of substantially bend portions; wherein adjacent rows of the plurality of parallel circumferential rows are coupled together by a singular bridging member extending from a substantially bend portion in a first row to a corresponding substantially bend portion in an adjacent second row, and wherein the bridging member coupling any two adjacent rows is circumferentially offset from a bridging member coupling any following two adjacent rows.

EXAMPLE 26: The expandable sheath of any examples herein, particularly example 25, wherein each substantially straight portion of the plurality of substantially straight portions being of equal lengths.

EXAMPLE 27: The expandable sheath of any examples herein, particularly examples 25 or 26, wherein the plurality of substantially straight portions comprise one or more portions having unequal lengths at the distal and/or proximal ends of the frame.

EXAMPLE 28: The expandable sheath of any examples herein, particularly examples 25-27, wherein the plurality of substantially bend portions in each row of the plurality of parallel circumferential rows define a bisecting line extending through an apex of each substantially bend portion of the plurality of substantially bend portions, the bisecting line extending longitudinally along a length of the sheath, and wherein each substantially straight portion of the plurality of substantially straight portions has a first angle from the bisecting line from 0° to about 90°.

EXAMPLE 29: The expandable sheath of any examples herein, particularly example 28, wherein when the frame is in the expanded position, the first angle increases by greater than 0% to about 600% of its original value.

EXAMPLE 30: The expandable sheath of any examples herein, particularly examples 1-6, wherein the cylindrical frame is formed by a first plurality of struts arranged along a first edge of a singular vertical member and a second plurality of struts arranged along an opposite second edge of the singular vertical member, wherein each strut of the first plurality of struts is spaced from each other such that a first gap is formed between two adjacent struts in the first plurality of struts; wherein each strut of the second plurality of struts is spaced from each other such that a second gap is formed between two adjacent struts in the second plurality of struts; wherein each strut of the first plurality of struts is axially offset along the singular vertical member from each strut of the second plurality of struts; and wherein in the unexpanded position, each strut of the first plurality of struts is disposed within the second gap, and each strut of the second plurality of struts is disposed within the first gap.

EXAMPLE 31: The expandable sheath of any examples herein, particularly example 30, wherein a width of the vertical member is less than 25% of a circumference of the cylindrical frame in the unexpanded position.

EXAMPLE 32: The expandable sheath of any examples herein, particularly example 30, wherein a width of the vertical member is less than 20% of a circumference of the cylindrical frame in the unexpanded position.

EXAMPLE 33: The expandable sheath of any examples herein, particularly example 30, wherein a width of the vertical member is less than 15% of a circumference of the cylindrical frame in the unexpanded position.

EXAMPLE 34: The expandable sheath of any examples herein, particularly example 30, wherein a width of the vertical member is less than 10% of a circumference of the cylindrical frame in the unexpanded position.

EXAMPLE 35: The expandable sheath of any examples herein, particularly example 30, wherein a width of the vertical member is less than 5% less of a circumference of the cylindrical frame in the unexpanded position.

EXAMPLE 36: The expandable sheath of any examples herein, particularly examples 30-35, wherein a width of the vertical member is measured along a portion of the circumference of the cylindrical frame corresponding to the vertical member.

EXAMPLE 37: The expandable sheath of any examples herein, particularly example 30, wherein a width of the vertical member is less than about 20% of a length of at least one strut of the first and second plurality of struts.

EXAMPLE 38: The expandable sheath of any examples herein, particularly example 30, wherein a width of the vertical member is less than about 15% of a length of at least one strut of the first and second plurality of struts.

EXAMPLE 39: The expandable sheath of any examples herein, particularly example 30, wherein a width of the vertical member is less than about 10% of a length of at least one strut of the first and second plurality of struts.

EXAMPLE 40: The expandable sheath of any examples herein, particularly example 30, wherein a width of the vertical member is less than about 5% of a length of at least one strut of the first and second plurality of struts.

EXAMPLE 41: The expandable sheath of any examples herein, particularly examples 37-40, wherein a length of at least one strut of the first and second plurality of struts is measured between a proximal end of the of the corresponding strut adjacent the vertical member, and a distal end of the corresponding strut.

EXAMPLE 42: The expandable sheath of any examples herein, particularly example 30, wherein a width of each strut of the first and second plurality of struts is less than about 25% of a circumference of the cylindrical frame in the unexpanded position.

EXAMPLE 43: The expandable sheath of any examples herein, particularly example 30, wherein a width of each strut of the first and second plurality of struts is less than about 20% of a circumference of the cylindrical frame in the unexpanded position.

EXAMPLE 44: The expandable sheath of any examples herein, particularly example 30, wherein a width of each strut of the first and second plurality of struts is less than about 15% of a circumference of the cylindrical frame in the unexpanded position. From 18 to 19

EXAMPLE 45: The expandable sheath of any examples herein, particularly example 30, wherein a width of each strut of the first and second plurality of struts is less than about 10% of a circumference of the cylindrical frame in the unexpanded position.

EXAMPLE 46: The expandable sheath of any examples herein, particularly example 30, wherein a width of each strut of the first and second plurality of struts is less than about 5% of a circumference of the cylindrical frame in the unexpanded position.

EXAMPLE 47: The expandable sheath of any examples herein, particularly examples 42-46, wherein the width of each strut of the first and second plurality of struts is measured in an axial direction along a longitudinal axis of the cylindrical frame.

EXAMPLE 48: The expandable sheath of any one of any examples herein,

particularly example 30-47, wherein a width of each strut of the first and second plurality of struts corresponds to a width of the vertical member.

EXAMPLE 49: The expandable sheath of any examples herein, particularly examples 30-47, wherein a width of each strut of the first and second plurality of struts is less than a width of the vertical member.

EXAMPLE 50: The expandable sheath of any examples herein, particularly examples 30-47, wherein a width of each strut of the first and second plurality of struts is greater than a width of the vertical member.

EXAMPLE 51: The expandable sheath of any examples herein, particularly examples 30-50, wherein a thickness of each strut of the first and second plurality of struts corresponds to a thickness of the vertical member.

EXAMPLE 52: The expandable sheath of any examples herein, particularly examples 30-50, wherein a thickness of each strut of the first and second plurality of struts is greater than a thickness of the vertical member.

EXAMPLE 53: The expandable sheath of any examples herein, particularly examples 30-51, wherein a thickness of each strut of the first and second plurality of struts is less than a thickness of the vertical member.

EXAMPLE 54: The expandable sheath of any examples herein, particularly examples 30-53, wherein a thickness of the first and second plurality of struts is measured radially between in an inner surface and corresponding outer surface of each strut of the first and second plurality of struts, wherein a thickness of the vertical member is measured radially between an inner and outer surface of the vertical member.

EXAMPLE 55: The expandable sheath of any examples herein, particularly example 30, a thickness of the vertical member is comparable to a thickness of the first and second plurality of struts.

EXAMPLE 56: The expandable sheath of any examples herein, particularly example 30, wherein the d₂ of the frame in the expanded position is between about 5 mm and about 10 mm.

EXAMPLE 57: The expandable sheath of any examples herein, particularly example 30, wherein d₂ of the frame in the expanded position is between about 5 mm and about 9 mm.

EXAMPLE 58: The expandable sheath of any examples herein, particularly example 30, wherein a width of the first gap and/or a width of the second gap is greater than a width of each strut of the first and second plurality of struts.

EXAMPLE 59: The expandable sheath of any examples herein, particularly example 30, wherein the frame is in the expanded position, at least a portion of the first plurality of struts remains at least partially disposed within the second gap and at least a portion of the second plurality of struts remains at least partially disposed within the first gap.

EXAMPLE 60: The expandable sheath of any examples herein, particularly example 30, wherein the frame is expanded, none of the first plurality of struts remains disposed within the second gap, and none of the second plurality of struts remains disposed within the first gap.

EXAMPLE 61: The expandable sheath of any examples herein, particularly examples 1-60, wherein the distal end of the frame comprises a pre-shaped flare that is configured to stay in a compressed state at the unexpanded position and to assume a flared configuration upon passing the medical device.

EXAMPLE 62: The expandable sheath of any examples herein, particularly example 61, wherein the pre-shaped flare is kept in the compressed state by a filament attached to one or more of the plurality of struts at the distal end of the frame and extending circumferentially around the plurality of struts at the distal end.

EXAMPLE 63: The expandable sheath of any examples herein, particularly example 62, wherein the distal end of the frame is in the expanded position, the filament stays attached to one or more of the plurality of struts at the distal end of the frame but does not extend circumferentially around the plurality of struts at the distal end allowing the distal end of the frame to flare radially outward.

EXAMPLE 64: The expandable sheath of any examples herein, particularly examples 1-63, wherein the first diameter d₁ is substantially uniform along the length of the frame.

EXAMPLE 65: The expandable sheath of any examples herein, particularly examples 1-64, wherein the first diameter d₁ varies along the length of the frame and wherein the first diameter d₁ at the proximal end is larger than the first diameter d₁ at the distal end.

EXAMPLE 66: The expandable sheath of any examples herein, particularly examples 1-65, wherein the frame is configured to contract to a diameter that is substantially the same as the first diameter d₁ upon removal of the medical device.

EXAMPLE 67: The expandable sheath of any examples herein, particularly examples 1-66, wherein the frame is configured to contract to a diameter that is from about 10% to about 90% larger than the first diameter d₁ upon removal of the medical device.

EXAMPLE 68: The expandable sheath of any examples herein, particularly examples 1-67, wherein the plurality of struts comprise a nitinol, stainless steel, cobalt-chrome alloys, polymer, or any combination thereof.

EXAMPLE 69: The expandable sheath of any examples herein, particularly examples 1-68, wherein at least a portion of the frame exhibits an elastic deformation.

EXAMPLE 70: The expandable sheath of any examples herein, particularly examples 1-69, wherein the sheath further comprises one or more polymeric layers.

EXAMPLE 71: The expandable sheath of any examples herein, particularly example 70, wherein the polymeric layer comprises one or more of polyethylene, polypropylene, polyether block amide (PEBAX®), silicon, or any combination thereof.

EXAMPLE 72: The expandable sheath of any examples herein, particularly examples 70-71, wherein at least a portion of the frame is embedded within the one or more polymeric layers.

EXAMPLE 73: The expandable sheath of any examples herein, particularly example 72, wherein the one or more polymeric layers comprise a plurality of longitudinally extending folds wherein the sheath is at the first diameter d₁.

EXAMPLE 74: The expandable sheath of any examples herein, particularly example 73, wherein the longitudinally extending folds create a plurality of circumferentially spaced ridges and a plurality of circumferentially spaced valleys.

EXAMPLE 75: The expandable sheath of any examples herein, particularly example 74, wherein, as the medical device passes through the sheath, the ridges and valleys level out to allow the sheath to radially expand.

EXAMPLE 76: The expandable sheath of any examples herein, particularly example 75, wherein the one or more polymeric layers have a first thickness wherein the sheath at the first diameter d₁.

EXAMPLE 77: The expandable sheath of any examples herein, particularly example 76, wherein, as the medical device passes through the sheath, the one or more polymeric layers are thinning out to a second thickness to allow the sheath to radially expand.

EXAMPLE 78: The expandable sheath of any examples herein, particularly examples 1-77, wherein the expandable cylindrical frame is configured to form a curve having a radius from about 1 inch to about 3 inches while maintaining the kink resistance.

EXAMPLE 79: The expandable sheath of any examples herein, particularly examples 1-78, wherein the sheath exhibits a decrease in an insertion force by greater than 0% to about 70%.

EXAMPLE 80: The expandable sheath of any examples herein, particularly examples 1-79, wherein the medical device is a cardiovascular device.

EXAMPLE 81: The expandable sheath of any examples herein, particularly example 80, wherein the cardiovascular device includes at least one of a prosthetic heart valve and a stent.

EXAMPLE 82: The expandable sheath of any examples herein, particularly examples 1-81, wherein the sheath further comprises an outer layer.

EXAMPLE 83: A method comprising arranging at least one plurality of struts to form an expandable cylindrical frame having a proximal and distal end, an inner surface and an outer surface, and a central axis extending along a length of the frame; wherein the expandable cylindrical frame has a first length and an opposite second length, and wherein in a first position at least a portion of the first length and at least a portion of the second length are substantially the same, wherein the expandable cylindrical frame is movable from the first position to a second position by curving away from the central axis, wherein at the second position at least a portion of the first length shortens and at least a portion of the second length elongates; wherein the inner surface of the expandable cylindrical frame defines a lumen configured to receive a medical device; and wherein the expandable cylindrical frame is configured to expand from a first diameter d₁ in an unexpanded position to a second diameter d₂ an expanded position upon passage of a medical device.

EXAMPLE 84: The method of any examples herein, particularly example 83, wherein the portion of the first length and the portion of the second length in the second position are located along a curved portion of the frame.

EXAMPLE 85: The method of any examples herein, particularly example 83 or 84, wherein the first length is provided on a first portion of an outer diameter of the formed expandable cylindrical frame and the second length is provided on a second portion of the outer diameter of the formed expandable cylindrical frame, wherein the first portion is circumferentially opposite to the second portion of the outer diameter.

EXAMPLE 86: The method of any examples herein, particularly examples 83-85, wherein the formed expandable cylindrical frame is kink-resistant.

EXAMPLE 87: The method of any examples herein, particularly examples 83-86, wherein the first diameter d₁ is from about 3 to about 5 mm.

EXAMPLE 88: The method of any examples herein, particularly examples 83-87, wherein the second diameter d₂ is from about 6 to about 10 mm.

EXAMPLE 89: The method of any examples herein, particularly examples 83-88, wherein the plurality of struts are arranged to form a zig-zag configuration comprising a plurality of substantially straight portions circumferentially followed by a plurality of substantially bend portions and wherein the zig-zag configuration is helically wound along a central axis to form the frame.

EXAMPLE 90: The method of any examples herein, particularly example 89, wherein each substantially straight portion of the plurality of substantially straight portions being of equal lengths.

EXAMPLE 91: The method of any examples herein, particularly examples 89 or 90, wherein the plurality of substantially straight portions comprise one or more portions having unequal lengths at the distal and/or proximal ends of the frame.

EXAMPLE 92: The method of any examples herein, particularly examples 83-91, wherein the plurality of substantially bend portions define a bisecting line extending through an apex of the plurality of substantially bend portions and extending longitudinally along a length of the sheath, and wherein each substantially straight portion of the plurality of substantially straight portions has a first angle from the bisecting line from 0° to about 90°.

EXAMPLE 93: The method of any examples herein, particularly example 92, wherein when the frame is in the expanded position, the first angle increases by greater than 0% to about 600% of its original value.

EXAMPLE 94: The method of any examples herein, particularly examples 83-88, wherein the plurality of struts are arranged in a zig-zag configuration to form a plurality of parallel circumferential rows, wherein each row of the plurality of parallel circumferential rows comprises a plurality of substantially straight portions circumferentially followed by a plurality of substantially bend portions; and wherein the plurality of parallel circumferential rows are axially coupled to each other by a singular vertical strut member disposed along a length of the frame.

EXAMPLE 95: The method of any examples herein, particularly example 94, wherein each substantially straight portion of the plurality of substantially straight portions being of equal lengths.

EXAMPLE 96: The method of any examples herein, particularly examples 94 or 95, wherein the plurality of substantially straight portions comprise one or more portions having unequal lengths at the distal and/or proximal ends of the frame.

EXAMPLE 97: The method of any examples herein, particularly examples 94-96, wherein the plurality of substantially bend portions define a bisecting line extending through an apex of the plurality of substantially bend portions and extending longitudinally along a length of the sheath, and wherein each substantially straight portion of the plurality of substantially straight portions has a first angle from the bisecting line from 0° to about 90°.

EXAMPLE 98: The method of any examples herein, particularly example 97, wherein when the frame is in the expanded position, the first angle increases by greater than 0% to about 600% of its original value.

EXAMPLE 99: The method of any examples herein, particularly examples 94-98, wherein the singular vertical strut member extends longitudinally along a length of the sheath and connects apexes of the substantially bending portions of each row of the plurality of parallel circumferential rows, the vertical strut member forming a second angle with the corresponding adjacent substantially straight portions from 0° to about 90°.

EXAMPLE 100: The method of any examples herein, particularly examples 94-99, wherein upon introduction of the medical device, the frame expands circumferentially in a direction opposite to the single vertical strut member.

EXAMPLE 101: The method of any examples herein, particularly examples 94-100, wherein upon introduction of the medical device, the frame does not expand along the singular vertical strut member.

EXAMPLE 102: The method of any examples herein, particularly examples 83-88, wherein the plurality of struts are arranged in a configuration wherein each strut of the plurality of struts is able to individually elongate in a circumferential direction along the length of the frame upon introduction of the medical device.

EXAMPLE 103: The method of any examples herein, particularly example 102, wherein the plurality of struts are arranged in a plurality of parallel axial rows, and wherein each row of the plurality of parallel axial rows comprises an undulating pattern unit, wherein each unit comprises: a first valley having a substantially straight bottom portion, wherein the substantially straight bottom portion of the first valley has a first width, wherein the first valley is followed by a first apex, a second valley, a second apex, and a third valley, and wherein a total width of the second and third valleys and the second apex is substantially the same as the first width of the substantially straight bottom portion of the first valley.

EXAMPLE 104: The method of any examples herein, particularly example 103, wherein the third valley is followed by a third apex and a first valley of a next undulating pattern unit in the same row.

EXAMPLE 105: The method of any examples herein, particularly example 103 or 104, wherein the plurality of parallel axial rows are arranged such that a second apex in each repetitive pattern unit of each row is positioned above and is coupled with at least a portion of a substantially straight bottom portion of a first valley of an adjacent row by a bridging member.

EXAMPLE 106: The method of any examples herein, particularly example 105, wherein the bridging member comprise a fastener.

EXAMPLE 107: The method of any examples herein, particularly examples 83-88, wherein the plurality of struts are arranged in a zig-zag configuration to form a plurality of parallel circumferential rows, wherein each row of the plurality of parallel circumferential rows comprises a plurality of substantially straight portions circumferentially followed by a plurality of substantially bend portions; wherein adjacent rows of the plurality of parallel circumferential rows are coupled together by a singular bridging member extending from a substantially bend portion in a first row to a corresponding substantially bend portion in an adjacent second row, and wherein the bridging member coupling any two adjacent rows is circumferentially offset from a bridging member coupling any following two adjacent rows.

EXAMPLE 108: The method of any examples herein, particularly example 107, wherein each substantially straight portion of the plurality of substantially straight portions being of equal lengths.

EXAMPLE 109: The method of any examples herein, particularly example 108, wherein the plurality of substantially straight portions comprise one or more portions having unequal lengths at the distal and/or proximal ends of the frame.

EXAMPLE 110: The method of any examples herein, particularly examples 107-109, wherein the plurality of substantially bend portions in each row of the plurality of parallel circumferential rows define a bisecting line extending through an apex of each substantially bend portion of the plurality of substantially bend portions, the bisecting line extending longitudinally along a length of the sheath, and wherein each substantially straight portion of the plurality of substantially straight portions has a first angle from the bisecting line from 0° to about 90°.

EXAMPLE 111: The method of any examples herein, particularly example 110, wherein when the frame is in the expanded position, the first angle increases by greater than 0% to about 600% of its original value.

EXAMPLE 112: The method of any examples herein, particularly examples 83-111, wherein a cylindrical frame is formed by a first plurality of struts arranged along a first edge of a singular vertical member and a second plurality of struts arranged along an opposite second edge of the singular vertical member, wherein each strut of the first plurality of struts is spaced from each other such that a first gap is formed between two adjacent struts in the first plurality of struts; wherein each strut of the second plurality of struts is spaced from each other such that a second gap is formed between two adjacent struts in the second plurality of struts; wherein each strut of the first plurality of struts is axially offset along the singular vertical member from each strut of the second plurality of struts; and wherein in the rest position, each strut of the first plurality of struts is disposed within the second gap, and each strut of the second plurality of struts is disposed within the first gap.

EXAMPLE 113: The method of any examples herein, particularly example 112, wherein a width of the vertical member is less than about 25% of a circumference of the cylindrical frame in the unexpanded position.

EXAMPLE 114: The method of any examples herein, particularly example 112, wherein a width of the vertical member is less than 20% of a circumference of the cylindrical frame in the unexpanded position.

EXAMPLE 115: The method of any examples herein, particularly example 112, wherein a width of the vertical member is less than 15% of a circumference of the cylindrical frame in the unexpanded position.

EXAMPLE 116: The method of any examples herein, particularly example 112, wherein a width of the vertical member is less than 10% of a circumference of the cylindrical frame in the unexpanded position.

EXAMPLE 117: The method of any examples herein, particularly example 112, wherein a width of the vertical member is less than 5% less of a circumference of the cylindrical frame in the unexpanded position.

EXAMPLE 118: The method of any examples herein, particularly examples 112-117, wherein the width of the vertical member is measured along a portion of the circumference of the cylindrical frame corresponding to the vertical member.

EXAMPLE 119: The method of any examples herein, particularly example 112, wherein a width of the vertical member is less than about 20% of a length of at least one strut of the first and second plurality of struts.

EXAMPLE 120: The method of any examples herein, particularly example 112, wherein a width of the vertical member is less than about 15% of a length of at least one strut of the first and second plurality of struts.

EXAMPLE 121: The method of any examples herein, particularly example 112, wherein a width of the vertical member is less than about 10% of a length of at least one strut of the first and second plurality of struts.

EXAMPLE 122: The method of any examples herein, particularly example 112, wherein a width of the vertical member is less than about 5% of a length of at least one strut of the first and second plurality of struts.

EXAMPLE 123: The method of any examples herein, particularly examples 119-122, wherein a length of at least one strut of the first and second plurality of struts is measured between a proximal end of the of the corresponding strut adjacent the vertical member, and a distal end of the corresponding strut.

EXAMPLE 124: The method of any examples herein, particularly example 112, wherein a width of each strut of the first and second plurality of struts is less than about 25% of a circumference of the cylindrical frame in the unexpanded position.

EXAMPLE 125: The method of any examples herein, particularly example 112, wherein a width of each strut of the first and second plurality of struts is less than about 20% of a circumference of the cylindrical frame in the unexpanded position.

EXAMPLE 126: The method of any examples herein, particularly example 112, wherein a width of each strut of the first and second plurality of struts is less than about 15% of a circumference of the cylindrical frame in the unexpanded position.

EXAMPLE 127: The method of any examples herein, particularly example 112, wherein a width of each strut of the first and second plurality of struts is less than about 10% of a circumference of the cylindrical frame in the unexpanded position.

EXAMPLE 128: The method of any examples herein, particularly example 112, wherein a width of each strut of the first and second plurality of struts is less than about 5% of a circumference of the cylindrical frame in the unexpanded position.

EXAMPLE 129: The method of any examples herein, particularly examples 124-128, wherein the width of each strut of the first and second plurality of struts is measured in an axial direction along a longitudinal axis of the cylindrical frame.

EXAMPLE 130: The method of any one of any examples herein, particularly examples 112-129, wherein a width of each strut of the first and second plurality of struts corresponds to a width of the vertical member.

EXAMPLE 131: The method of any examples herein, particularly examples 112-130, wherein a width of each strut of the first and second plurality of struts is less than a width of the vertical member.

EXAMPLE 132: The method of any examples herein, particularly examples 112-130, wherein a width of each strut of the first and second plurality of struts is greater than a width of the vertical member.

EXAMPLE 133: The method of any examples herein, particularly examples 112-132, wherein a thickness of each strut of the first and second plurality of struts corresponds to a thickness of the vertical member.

EXAMPLE 134: The method of any examples herein, particularly examples 112-132, wherein a thickness of each strut of the first and second plurality of struts is greater than a thickness of the vertical member.

EXAMPLE 135: The method of any examples herein, particularly examples 112-132, wherein a thickness of each strut of the first and second plurality of struts is less than a thickness of the vertical member.

EXAMPLE 136: The method of any examples herein, particularly examples 112-135, wherein a thickness of the first and second plurality of struts is measured radially between in an inner surface and corresponding outer surface of each strut of the first and second plurality of struts, wherein the thickness of the vertical member is measured radially between an inner and outer surface of the vertical member.

EXAMPLE 137: The method of any examples herein, particularly example 112, wherein a thickness of the vertical member is comparable to a thickness of the first and second plurality of struts.

EXAMPLE 138: The method of any examples herein, particularly example 112, wherein d₂ of the frame in the expanded position is between about 5 mm and about 10 mm.

EXAMPLE 139: The method of any examples herein, particularly example 112, wherein d₂ of the frame in the expanded position is between 5 mm and 9 mm.

EXAMPLE 140: The method of any examples herein, particularly example 112, wherein a width of the first gap and/or a width of the second gap is greater than a width of each strut of the first and second plurality of struts.

EXAMPLE 141: The method of any examples herein, particularly examples 112-140, wherein the frame is in the expanded position, at least a portion of the first plurality of struts remains at least partially disposed within the second gap and at least a portion of the second plurality of struts remains at least partially disposed within the first gap.

EXAMPLE 142: The method of any examples herein, particularly example 141, wherein the frame is expanded, none of the first plurality of struts remains disposed within the second gap, and none of the second plurality of struts remains disposed within the first gap.

EXAMPLE 143: The method of any examples herein, particularly examples 83-142, wherein further forming a pre-shaped flare at the distal end of the frame such that the pre-shaped flare is configured to stay compressed at the rest position and to assume a flared configuration upon passing the medical device.

EXAMPLE 144: The method of any examples herein, particularly example 143, wherein the pre-shaped flare is kept compressed by attaching a filament one or more of the plurality of struts at the distal end of the frame and extending it circumferentially around the plurality of struts at the distal end.

EXAMPLE 145: The method of any examples herein, particularly example 144, wherein the distal end of the frame is in the expanded position the filament is configured to stay attached to one or more of the plurality of struts at the distal end of the frame without extending circumferentially around the plurality of struts at the distal end thereby allowing the distal end of the frame to flare radially outward.

EXAMPLE 146: The method of any examples herein, particularly examples 83-145, wherein the first diameter d₁ is substantially uniform along the length of the frame.

EXAMPLE 147: The method of any examples herein, particularly examples 83-146, wherein the first diameter d₁ varies along the length of the frame and wherein the first diameter d₁ at the proximal end is larger than the first diameter d₁ at the distal end.

EXAMPLE 148: The method of any examples herein, particularly examples 83-147, wherein the frame is configured to contract to a diameter that is substantially the same as the first diameter d₁ upon removal of the medical device.

EXAMPLE 149: The method of any examples herein, particularly examples 83-148, wherein the frame is configured to contract to a diameter that is from about 10 to about 90% larger than the first diameter d₁ upon removal of the medical device.

EXAMPLE 150: The method of any examples herein, particularly examples 83-149, wherein the plurality of struts comprise a nitinol, stainless steel, cobalt-chrome alloy, polymer, or any combination thereof.

EXAMPLE 151: The method of any examples herein, particularly examples 83-150, wherein at least a portion of the frame exhibits an elastic deformation.

EXAMPLE 152: The method of any examples herein, particularly examples 83-151, further comprising applying one or more polymeric layers to the expandable cylindric frame.

EXAMPLE 153: The method of any examples herein, particularly example 152, wherein the polymeric layer comprises one or more of polyethylene, polypropylene, polyether block amide (PEBAX®), silicon, or any combination thereof.

EXAMPLE 154: The method of any examples herein, particularly examples 152-153, wherein at the step of applying, at least a portion of the frame is embedded within the one or more polymeric layers.

EXAMPLE 155 The method of any examples herein, particularly example 154, wherein the one or more polymeric layers comprise a plurality of longitudinally extending folds wherein the sheath is at the first diameter d₁.

EXAMPLE 156: The method of any examples herein, particularly example 155, wherein the longitudinally extending folds create a plurality of circumferentially spaced ridges and a plurality of circumferentially spaced valleys.

EXAMPLE 157: The method of any examples herein, particularly example 156, wherein, as the medical device passes through the sheath, the ridges and valleys level out to allow the sheath to radially expand.

EXAMPLE 158: The method of any examples herein, particularly example 157, wherein the one or more polymeric layers have a first thickness wherein the sheath is at the first diameter d₁.

EXAMPLE 159: The method of any examples herein, particularly example 158, wherein, as the medical device passes through the sheath, the one or more polymeric layers are thinning out to a second thickness to allow the sheath to radially expand.

EXAMPLE 160: The method of any examples herein, particularly examples 83-159, wherein the expandable cylindrical frame is configured to form a curve having a radius from about 1 inch to about 3 inches while maintaining the kink resistance.

EXAMPLE 161: The method of any examples herein, particularly examples 83-160, wherein the sheath exhibits a decrease in an insertion force by greater than 0% to about 70%.

EXAMPLE 162: The method of any examples herein, particularly examples 83-161, wherein the medical device is a cardiovascular device.

EXAMPLE 163: The method of any examples herein, particularly example 162, wherein the cardiovascular device includes at least one of a prosthetic heart valve and a stent.

EXAMPLE 164: The method of any examples herein, particularly examples 83-163, further comprising applying an outer layer on the sheath.

Although several aspects of the disclosure have been disclosed in the foregoing specification, it is understood by those skilled in the art that many modifications and other aspects of the disclosure will come to mind to which the disclosure pertains, having the benefit of the teaching presented in the foregoing description and associated drawings. It is thus understood that the disclosure is not limited to the specific aspects disclosed hereinabove and that many modifications and other aspects are intended to be included within the scope of the appended claims. Moreover, although specific terms are employed herein, as well as in the claims which follow, they are used only in a generic and descriptive sense and not for the purposes of limiting the described disclosure nor the claims which follow. We, therefore, claim as our disclosure all that comes within the scope and spirit of these claims.

1. An expandable sheath for deploying a medical device comprising:
at least one plurality of struts arranged to form an expandable cylindrical frame having a proximal and distal end, an inner surface and an outer surface, and a central axis extending along a length of the frame;
wherein the expandable cylindrical frame has a first length and an opposite second length, and wherein, in a first position, at least a portion of the first length and at least a portion of the second length are substantially the same;
wherein the expandable cylindrical frame is movable from the first position to a second position by curving away from the central axis, wherein at the second position at least a portion of the first length shortens and at least a portion of the second length elongates;
wherein the inner surface of the expandable cylindrical frame defines a lumen configured to receive a medical device;
wherein the expandable cylindrical frame is configured to expand from a first diameter d1 in an unexpanded position to a second diameter d2 in an expanded position upon passage of a medical device; and
wherein the expandable cylindrical frame is kink-resistant.

2. The expandable sheath of claim 1, wherein the portion of the first length and the portion of the second length in the second position are located along a curved portion of the frame.

3. The expandable sheath of claim 1, wherein the first length is provided on a first portion of an outer diameter of the expandable cylindrical frame and the second length is provided on a second portion of the outer diameter of the expandable cylindrical frame, wherein the first portion is circumferentially opposite to the second portion of the outer diameter.

4. The expandable sheath of claim 1, wherein the plurality of struts are arranged to form a zig-zag configuration comprising a plurality of substantially straight portions circumferentially followed by a plurality of substantially bend portions and wherein the zig-zag configuration is wound along a central axis to form the frame.

5. The expandable sheath of claim 4, wherein the plurality of substantially bend portions define a bisecting line extending through an apex of the plurality of substantially bend portions and extending longitudinally along a length of the sheath, and wherein each substantially straight portion of the plurality of substantially straight portions has a first angle from the bisecting line from 0° to about 90°,
wherein, when the frame is in the expanded position, the first angle increases by an amount greater than 0% to about 600% of its original value.

6. The expandable sheath of claim 1, wherein the plurality of struts are arranged in a zig-zag configuration to form a plurality of parallel circumferential rows, wherein each row of the plurality of parallel circumferential rows comprises a plurality of substantially straight portions circumferentially followed by a plurality of substantially bend portions; and wherein the plurality of parallel circumferential rows are axially coupled to each other by a singular vertical strut member disposed along a length of the frame.

7. The expandable sheath of claim 6, wherein the plurality of substantially bend portions define a bisecting line extending through an apex of the plurality of substantially bend portions and extending longitudinally along a length of the sheath, and wherein each substantially straight portion of the plurality of substantially straight portions has a first angle from the bisecting line 0° to about 90°,
wherein, when the frame is in the expanded position, the first angle increases by an amount greater than 0% to about 600% of its original value.

8. The expandable sheath of claim 6, wherein the singular vertical strut member extends longitudinally along a length of the sheath and connects apexes of the substantially bend portions of each row of the plurality of parallel circumferential rows, and the vertical strut member forms a second angle with each substantially straight portion of the corresponding adjacent substantially straight portions of 0° to about 90°,
wherein during expansion the frame expands circumferentially in a direction opposite to the single vertical strut member, and wherein the frame does not expand along the singular vertical strut member.

9. The expandable sheath of claim 1, wherein the plurality of struts are arranged in a configuration wherein each strut of the plurality of struts is able to individually elongate in a circumferential direction along the length of the frame upon introduction of the medical device.

10. The expandable sheath of claim 9, wherein the plurality of struts is arranged in a plurality of parallel axial rows, each row of the plurality of parallel axial rows comprises an undulating pattern unit,
wherein each unit comprises a first valley having a substantially straight bottom portion, wherein the substantially straight bottom portion of the first valley has a first width,
wherein the first valley is followed by a first apex, a second valley, a second apex, and a third valley, and
wherein a total width of the second and third valleys and the second apex is substantially the same as the first width of the substantially straight bottom portion of the first valley,
wherein the third valley is followed by a third apex and a first valley of a next undulating pattern unit in the same row.

11. The expandable sheath of claim 10, wherein the plurality of parallel axial rows are arranged such that a second apex in each repetitive pattern unit of each row is positioned above and is coupled with at least a portion of a substantially straight bottom portion of a first valley of an adjacent row by a bridging member.

12. The expandable sheath of claim 1, wherein the plurality of struts are arranged in a zig-zag configuration to form a plurality of parallel circumferential rows, wherein each row of the plurality of parallel circumferential rows comprises a plurality of substantially straight portions circumferentially followed by a plurality of substantially bend portions; wherein adjacent rows of the plurality of parallel circumferential rows are coupled together by a singular bridging member extending from a substantially bend portion in a first row to a corresponding substantially bend portion in an adjacent second row, and wherein the singular bridging member coupling any two adjacent rows is circumferentially offset from a bridging member coupling any following two adjacent rows.

13. The expandable sheath of claim 12, wherein the plurality of substantially bend portions in each row of the plurality of parallel circumferential rows define a bisecting line extending through an apex of each substantially bend portion of the plurality of substantially bend portions, the bisecting line extending longitudinally along a length of the sheath, and wherein each substantially straight portion of the plurality of substantially straight portions has a first angle from the bisecting line from 0° to about 90°,
wherein, when the frame is in the expanded position, the first angle increases by an amount greater than 0% to about 600% of its original value.

14. The expandable sheath of claim 1, wherein the cylindrical frame is formed by a first plurality of struts arranged along a first edge of a singular vertical member and a second plurality of struts arranged along an opposite second edge of the singular vertical member,
wherein each strut of the first plurality of struts is spaced from each other such that a first gap is formed between two adjacent struts in the first plurality of struts;
wherein each strut of the second plurality of struts is spaced from each other such that a second gap is formed between two adjacent struts in the second plurality of struts;
wherein each strut of the first plurality of struts is axially offset along the singular vertical member from each strut of the second plurality of struts; and
wherein in the unexpanded position, each strut of the first plurality of struts is disposed within the second gap, and each strut of the second plurality of struts is disposed within the first gap.

15. The expandable sheath of claim 14, wherein a width of the first gap and/or a width of the second gap is greater than a width of each strut of the first and second plurality of struts, wherein the frame is in the expanded position, at least a portion of the first plurality of struts remains at least partially disposed within the second gap and at least a portion of the second plurality of struts remains at least partially disposed within the first gap.

16. The expandable sheath of claim 1, wherein the distal end of the frame comprises a pre-shaped flare that is configured to stay in a compressed state at the unexpanded position and to assume a flared configuration upon passing the medical device.

17. The expandable sheath of claim 16, wherein the pre-shaped flare is kept in the compressed state by a filament attached to one or more of the plurality of struts at the distal end of the frame and extending circumferentially around the plurality of struts at the distal end,
wherein, when the distal end of the frame is in the expanded position, the filament stays attached to one or more of the plurality of struts at the distal end of the frame and does not extend circumferentially around the plurality of struts at the distal end thereby allowing the distal end of the frame to flare radially outward.

18. The expandable sheath of claim 1, wherein the frame is configured to contract to a diameter that is from about 10% to about 90% larger than the first diameter d1 upon removal of the medical device.

19. The expandable sheath of claim 1, wherein the sheath further comprises one or more polymeric layers,
wherein at least a portion of the frame is embedded within the one or more polymeric layers,
wherein the one or more polymeric layers comprise a plurality of longitudinally extending folds wherein the sheath is at the first diameter d₁, and wherein the longitudinally extending folds create a plurality of circumferentially spaced ridges and a plurality of circumferentially spaced valleys,
wherein, as the medical device passes through the sheath, the ridges and valleys level out to allow the sheath to radially expand,
wherein the one or more polymeric layers have a first thickness wherein the sheath at the first diameter d₁, and wherein, as the medical device passes through the sheath, the one or more polymeric layers are thinning out to a second thickness to allow the sheath to radially expand.

20. The expandable sheath of claim 1, wherein the sheath exhibits a decrease in an insertion force by greater than 0% to about 70%.
